(19) United States
(12) Patent Application Publication
(10) Pub. No.: US 2024/0157041 A1
(43) Pub. Date: May 16, 2024

(54) SYSTEMS FOR ASPIRATING AND IRRIGATING BODY CAVITIES, AND ASSOCIATED DEVICES AND METHODS

(71) Applicant: Inari Medical, Inc., Irvine, CA (US)

(72) Inventors: Christopher Andrew Zikry, Placentia, CA (US); Steven McConnell, Anaheim, CA (US)

(21) Appl. No.: 18/506,892

(22) Filed: Nov. 10, 2023

Related U.S. Application Data
(60) Provisional application No. 63/383,440, filed on Nov. 11, 2022.
(60) Provisional application No. 63/426,560, filed on Nov. 18, 2022.

(51) Int. Cl. A61M 3/02; A61M 1/00; A61M 25/00; A61M 39/24

(52) U.S. Cl. CPC A61M 3/0283; A61M 1/77; A61M 3/0262; A61M 25/007; A61M 39/24; A61M 2025/0039

(57) ABSTRACT

Disclosed herein are systems for aspirating, irrigating, and/or mechanically disrupting body cavities, such as abdominal abscesses, empyemas, and/or (e.g., complicated) pleural effusions, and associated devices and methods. In some embodiments, an aspiration and irrigation system includes an inner catheter defining an aspiration lumen and an outer tube positioned coaxially around the inner catheter and defining an irrigation lumen. A distal portion of the outer tube can be bonded to the inner catheter, and a plurality of apertures can be formed through the outer tube proximal of the bond. The aspiration lumen can be fluidly coupled to an aspiration circuit configured to aspirate through the aspiration lumen, and the irrigation lumen can be fluidly coupled to an irrigation circuit configured to flow an irrigation fluid through the irrigation lumen and out of the apertures.

### CROSS-REFERENCE TO RELATED APPLICATIONS

This application claims the benefit of U.S. Provisional Patent Application No. 63/383,440, filed Nov. 11, 2022, and titled “SYSTEMS FOR ASPIRATING AND IRRIGATING BODY CAVITIES, AND ASSOCIATED DEVICES AND METHODS,” and U.S. Provisional Patent Application No. 63/426,560, filed Nov. 18, 2022, and titled “SYSTEMS FOR ASPIRATING AND IRRIGATING BODY CAVITIES, AND ASSOCIATED DEVICES AND METHODS,” each which is incorporated herein by reference in its entirety.

### TECHNICAL FIELD

The present technology generally relates to systems for aspirating, irrigating, and/or mechanically disrupting material within body cavities, such as abdominal abscesses, empyemas, and/or (e.g., complicated) pleural effusions.

### BACKGROUND

Abdominal abscesses and pleural effusions are collections that fill a body cavity with low viscosity, sterile serosanguinous fluid. If the collection is large enough, percutaneous drainage can be performed to drain the collection contents. The drainage is typically performed with the use of an indwelling percutaneous drainage catheter which is typically left in for less than a week for simple collections. Upon further progression of the disease state, the pleural effusion can become a complicated pleural/parapneumonic effusion or pleural empyema, both of which involve infection of the cavity contents. Intraabdominal abscesses can also become infected, leading to a more complicated presentation.

Such complicated abdominal abscess collections, pleural effusions, and pleural empyemas can generate thick, viscous pus (purulent fluid) with the inclusion of necrotic debris, blood clots, enteral content, and/or multiple loculations. Loculations are contained within fibrinous sheets (e.g., septations) that create distinct fluid-filled pockets within a single cavity. The progression of these disease states to a complicated presentation significantly challenges the ability for current percutaneous drains to evacuate the collection efficiently and completely. For example, current indwelling drains are limited by inner lumen size, drainage side-hole diameter, number of drainage side-holes, constrictions along the fluid path (e.g., stopcock), length of the fluid path, and the pressure difference between the inlet (abscess) and outlet (collection bag). Although drains have improved in size, shape, and suction over time, current technology still struggles to evacuate complex collections. This is evidenced by the need for multiple drains within the same collection, the need for multiple drain replacements due to clogging or malpositioning, long duration of drainage (days to months), the use of pharmacologic agents to supplement drainage efficiency, and the use of off-label devices to make collections more amenable to percutaneous drainage.

Some thrombectomy devices have been used off-label for mechanical debridement of cavities. These devices are not targeted and/or lack precise spatial control. Additionally, pharmacological agents such as tissue plasminogen activator (tPA) and deoxyribonuclease (DNase) have been administered to liquefy the collection and accelerate drainage by reducing viscosity of the fluid; however, most protocols are labor-intensive and time-consuming.

Further, drainage catheters require daily flushing with a low volume of sterile saline to prevent the catheter from clogging and impeding flow. While flushing aims to clear the catheter and maintain its patency, irrigation is meant to mobilize debris and reduce viscosity of local contents within the collection. Irrigation techniques have been performed using current drainage catheters. For such techniques, a large volume of sterile saline is flushed through a placed percutaneous drain and immediately aspirated. This method can also effectively clear a clogged drain, but advances debris already within the drain back into the cavity, thus potentially allowing it to later clog the drain again. Sometimes, the use of two separate drains in a single cavity can reduce clogging after irrigation.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1A is a partially schematic side view of an aspiration and irrigation system in accordance with embodiments of the present technology.

FIG. 1B is an enlarged side perspective view of a distal portion of a catheter assembly of the system shown in FIG. 1A in accordance with embodiments of the present technology.

FIG. 1C is an enlarged distally-facing perspective view of a distal portion of the catheter assembly of FIG. 1A in accordance with embodiments of the present technology.

FIGS. 2A and 2B are a side view and an enlarged partially-schematic side view of a pressure source assembly and an irrigation assembly of the system of FIG. 1A in accordance with embodiments of the present technology.

FIGS. 3A-3C are enlarged side views of a mechanical disruptor assembly in a compressed position, a partially-expanded position, and an expanded position, respectively, that is configured to be advanced through the catheter assembly of FIG. 1A to within a body cavity to mechanically disrupt material therein in accordance with embodiments of the present technology.

FIG. 3D is a side view of a handle of the mechanical disruptor assembly of FIGS. 3A-3C in accordance with embodiments of the present technology.

FIG. 4 is a flow diagram of a process or method for treating a body cavity of a patient using the system of FIG. 1A in accordance with embodiments of the present technology.

FIGS. 5A-5E are side cross-sectional views of a distal portion of the catheter assembly of FIG. 1A during different stages of the method of FIG. 4 in accordance with embodiments of the present technology.

FIG. 6 is a side view of an aspiration and irrigation system in accordance with additional embodiments of the present technology.

FIG. 7A is a side view of a catheter assembly of the system of FIG. 6 in accordance with embodiments of the present technology.

FIG. 7B is a side view of the of the catheter assembly of FIG. 7A with a dilator inserted therein in accordance with embodiments of the present technology.

FIGS. 8A and 8B are enlarged side views of a distal portion of an elongate member of the catheter assembly of FIG. 6 in accordance with embodiments of the present technology.

### DETAILED DESCRIPTION

The present technology is generally directed to systems for aspirating, irrigating, and/or mechanically disrupting material/contents within body cavities, such as abdominal abscesses, empyemas, and/or (e.g., complicated) pleural effusions, and associated devices and methods. In some embodiments, an aspiration and irrigation system configured in accordance with the present technology includes an inner catheter defining an aspiration lumen and an outer tube positioned coaxially around the inner catheter and defining an irrigation lumen. A distal portion of the outer tube can be fluidly sealed to the inner catheter, and a plurality of apertures can be formed through the outer tube proximal to the fluidly sealed portion. The aspiration lumen can be fluidly coupled to an aspiration circuit configured to aspirate the aspiration lumen, and the irrigation lumen can be fluidly coupled to an irrigation circuit configured to flow an irrigation fluid through the irrigation lumen and out of the apertures. The catheter assembly can be positioned within a body cavity, and the aspiration circuit can be operated to aspirate material from the abscess. At the same or a different time, the irrigation circuit can be operated to irrigate the cavity with the irrigation fluid to, for example, break apart (e.g., mobilize) material within the cavity and/or reduce the viscosity of the material within the cavity.

In some aspects of the present technology, the aspiration and irrigation system (i) maximizes the area of aspiration lumen along the entire length of the aspiration lumen, (ii) provides vigorous circumferential irrigation to reduce viscosity of the cavity contents and break up loculations and other large debris, and (iii) separates the aspiration and irrigation circuits. The catheter assembly permits a physician to quickly irrigate and aspirate large, complex, collections to save drain management time and overcome repeated drain clogging. The complicated material can be evacuated with the aspiration and irrigation system during an initial treatment procedure, thus making the collection amenable to drainage with currently available drainage catheters. Alternatively, the aspiration and irrigation system may be employed in collections that currently available drainage catheters have failed to evacuate.

The aspiration and irrigation system can be designed to maximize flow of material by utilizing Poiseuille's law as defined below. In some embodiments, the pressure differential is maximized by using an aspiration source comprising a 60 cc syringe, which is capable of creating a vacuum of −25.5 inHg when fully evacuated. The aspiration catheter radius can be maximized by maintaining a single lumen having the same diameter from the distal tip of the aspiration catheter to the syringe by utilizing a large bore side port tubing and large bore syringe. Fluid viscosity can be lowered via the irrigation process which can dilute the cavity contents. The length of the system can be minimized by maintaining a minimal distance between the tip of the catheter and the aspiration source/syringe. In contrast, current drains typically use excess tubing length to connect to gravity collection bags, wall suction, or suction bulbs, which decreases the efficiency of the drain.

In cases where contents within the cavity are too viscous or large for the aspiration catheter, a mechanical element can be employed. The mechanical element can have a size and shape that can be safely controlled to a desired geometry and manipulated within the cavity to aid with subsequent aspiration and drainage.

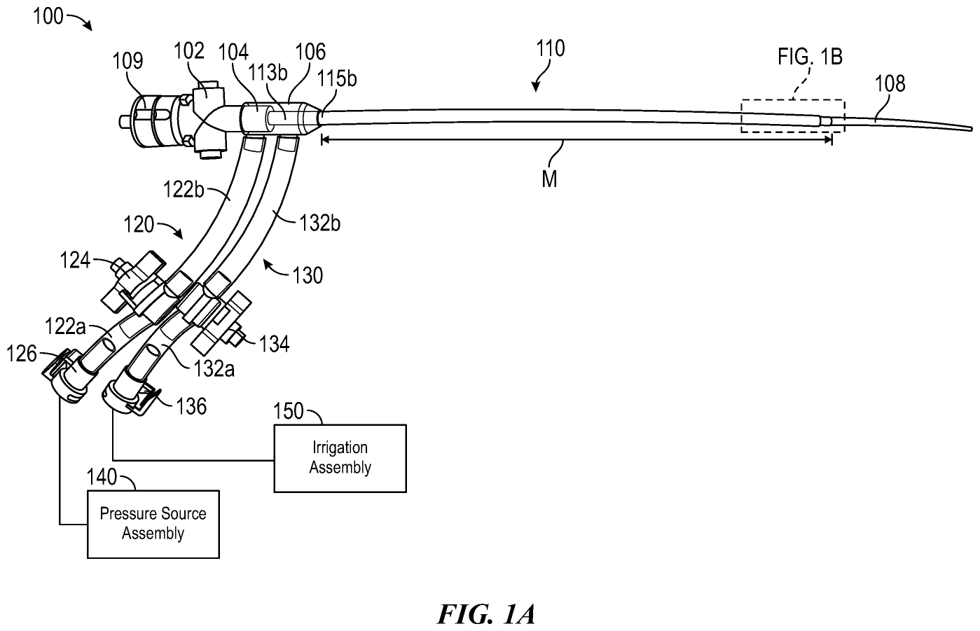

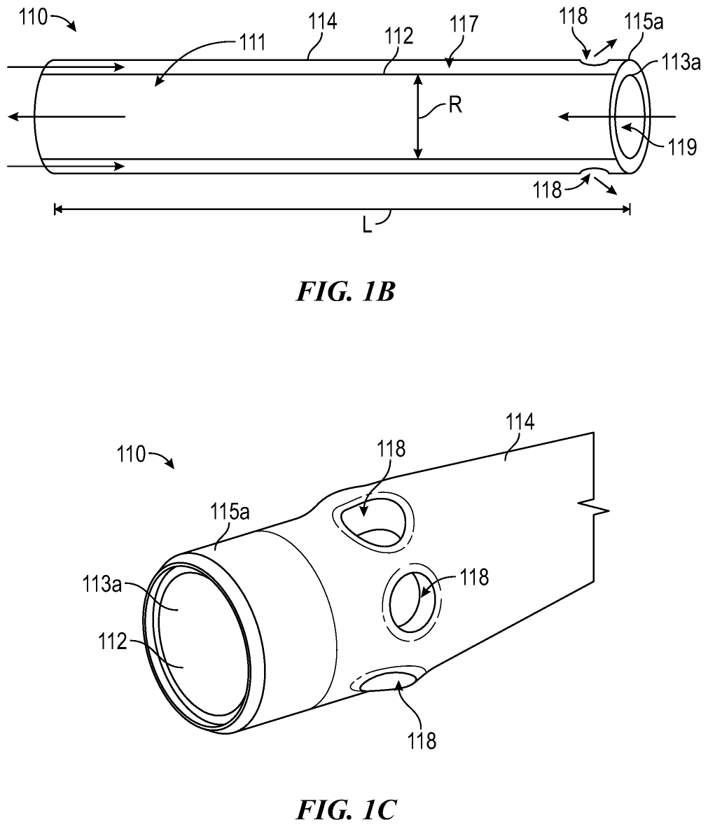

Certain details are set forth in the following description and in FIGS. 1-8B to provide a thorough understanding of various embodiments of the present technology. In other instances, well-known structures, materials, operations, and/or systems often associated with percutaneous procedures, body cavity material removal procedures, catheters, and the like are not shown or described in detail in the following disclosure to avoid unnecessarily obscuring the description of the various embodiments of the technology. Moreover, although reference is primarily made to aspiration and drainage catheters for use in removing material from body cavities, the catheters of the present technology can be other types of catheters and/or can be used in other types of medical procedures. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

As used herein, unless expressly indicated otherwise, the terms “about,” “approximately,” “substantially” and the like mean within plus or minus 10% of the stated value. To the extent any materials incorporated herein by reference conflict with the present disclosure, the present disclosure controls.

FIG. 1A is a partially schematic side view of an aspiration and irrigation system 100 (“system 100”) in accordance with embodiments of the present technology. In the illustrated embodiment, the system 100 includes a catheter assembly 110 fluidly coupled to (i) a valve 102, (ii) a first tubing assembly 120 via a first hub 104, and (iii) a second tubing assembly 130 via a second hub 106. The system 100 can include several features generally similar or identical to those of the clot treatment systems described in detail in U.S. patent application Ser. No. 16/536,185, filed August 8, 2019, and titled “SYSTEM FOR TREATING EMBOLISM AND ASSOCIATED DEVICES AND METHODS,” which is incorporated herein by reference in its entirety.

FIG. 1B is an enlarged side perspective view of a distal portion of the catheter assembly 110 shown in FIG. 1A in accordance with embodiments of the present technology. In the illustrated embodiment, the catheter assembly 110 and extends along an axis L (e.g., a longitudinal axis) includes an inner elongate member 112 (which can also be referred to as an inner sheath, an inner tube, an inner catheter, an aspiration member, an aspiration sheath, an aspiration tube, an aspiration catheter, and/or the like) and an outer elongate member 114 (which can also be referred to as an outer sheath, an outer tube, an outer catheter, an irrigation member, an irrigation sheath, an irrigation tube, an irrigation catheter, and/or the like) coaxially positioned at least partially around the inner elongate member 112. The outer elongate member 114 is shown as partially transparent in FIG. 1B for clarity. The inner elongate member 112 can be a reinforced thin-walled catheter. In some embodiments, the inner elongate member 112 can include some features that are at least generally similar in structure and function, or identical in structure and function, to those of the catheters disclosed in (i) U.S. patent application Ser. No. 17/529,018, filed Nov. 17, 2021, and titled “CATHETERS HAVING SHAPED DISTAL PORTIONS, AND ASSOCIATED SYSTEMS AND METHODS,” and/or (ii) U.S. patent application Ser. No. 17/529,064, filed Nov. 17, 2021, and titled “CATHETERS HAVING STEERABLE DISTAL PORTIONS, AND ASSOCIATED SYSTEMS AND METHODS,” each of which is incorporated herein by reference in its entirety. The outer elongate member 114 can be a tube formed from a plastic material, elastomeric material, and/or thermoplastic elastomer (TPE) material, such as a TPE manufactured by Arkema S.A., of Colombes, France, such as the TPEs manufactured under the trademark “Pebax.” In other embodiments, the outer elongate member 114 can be a reinforced thin-walled catheter. The outer elongate member 114 can have a size of between about 6-30 French, such as a size of 6 French, 8 French, 12 French, 16 French, 20 French, 24 French, 26 French, or 30 French. The inner elongate member 112 can have a size smaller than the outer elongate member 114, such a size between about 1-8 French smaller than the outer elongate member 114.

FIG. 1C is an enlarged distally-facing perspective view of the distal portion of the catheter assembly 110 in accordance with embodiments of the present technology. Referring to FIGS. 1B and 1C, a distal end portion 115a of the outer elongate member 114 can be fluidly sealed to/against (e.g., coupled to, mechanically attached to, bonded to, affixed to, etc.) to a distal portion 113a (e.g., a distal end portion) of the inner elongate member 112. Referring to FIG. 1A, a proximal end portion 113b of the inner elongate member 112 can be coupled to (e.g., bonded to) the first hub 104 and/or the valve 102, and a proximal end portion 115b of the outer elongate member 114 can be coupled to (e.g., bonded to) the second hub 106.

Referring to FIG. 1B, the inner elongate member 112 defines an inner lumen 111 (e.g., an aspiration lumen) and the outer elongate member 114 defines an outer lumen 117 (e.g., an irrigation lumen). The inner lumen 111 is accessible at the distal portion 113a of the inner elongate member 112 via a distal opening 119 (e.g., an aspiration opening). Referring to FIGS. 1B and 1C, the distal end portion 115a of the outer elongate member 114 includes/defines one or more (e.g., a plurality of) circumferentially distributed apertures 118 (e.g., holes) fluidly coupled/connected to the outer lumen 117. As described in further detail below, the apertures 118 can serve as an outlet for an irrigation fluid, a targeted drug, and/or another fluid to flow out of the outer lumen 117 to outside the catheter assembly 110. In the illustrated embodiment, the apertures 118 have a circular shape. The size of the apertures 118, the shape of the apertures 118, a clearance between the outer elongate member 114 and the inner elongate member 112, and/or a lumen diameter of the second tubing assembly 130 (FIG. 1A), including the tubing sections 132a-b and the fluid control device 134, can be controlled (e.g., selected) to permit vigorous infusion of an irrigation fluid through the apertures 118. Further, the number of the apertures 118, the size of the apertures 118, the shape of the apertures 118 (e.g., circular, square, rectangular, rectilinear, polygonal, irregular, etc.) can be adjusted to adjust a jet fluid angle of the irrigation fluid leaving the apertures 118. In some embodiments, the distal opening 119 extends in a plane orthogonal to the axis L, and the apertures 118 extend along planes different than that of the distal opening 119 (e.g., planes orthogonal to the distal opening 119). In some embodiments, the apertures 118 are fluidly coupled/connected to the outer lumen 117 at the distal end portion 115a of the outer elongate member 114 where the distal end portion 115a (fluidly coupled/connected portion) extends proximal such that there is a longer distance between the distal opening 119 and the apertures 118.

Referring to FIGS. 1A-1C, the inner lumen 111 is fluidly coupled to the first tubing assembly 120 via the first hub 104, and the outer lumen 117 is fluidly coupled to the second tubing assembly 130 via the second hub 106. The valve 102 is fluidly coupled to the inner lumen 111 of the inner elongate member 112. In some embodiments, the valve 102 is an actuated access valve that is configured to maintain fluid control during a body cavity treatment procedure by inhibiting or preventing fluid flow in the proximal direction through the valve 102 as various components such as delivery sheaths, pull members, guidewires, interventional devices, mechanical disruptor assemblies, other aspiration catheters, and so on, are inserted through the valve 102 to be delivered through the inner elongate member 112 to a treatment site in a body cavity. In some embodiments, the valve 102 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety.

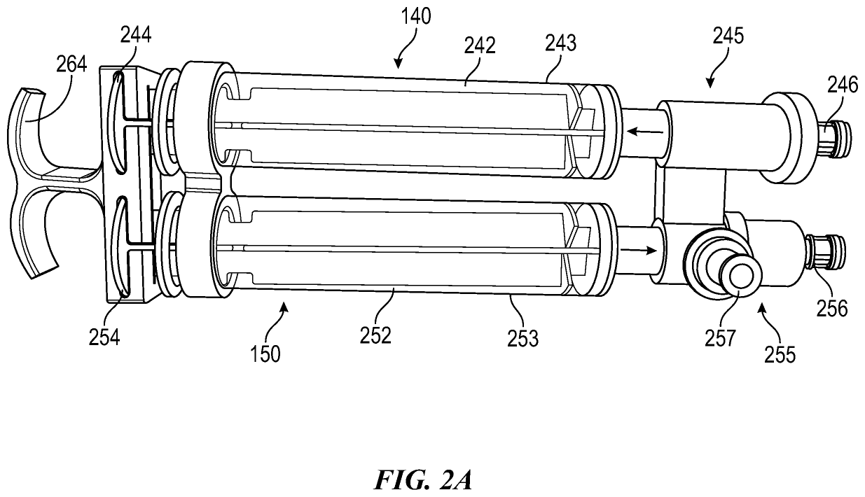

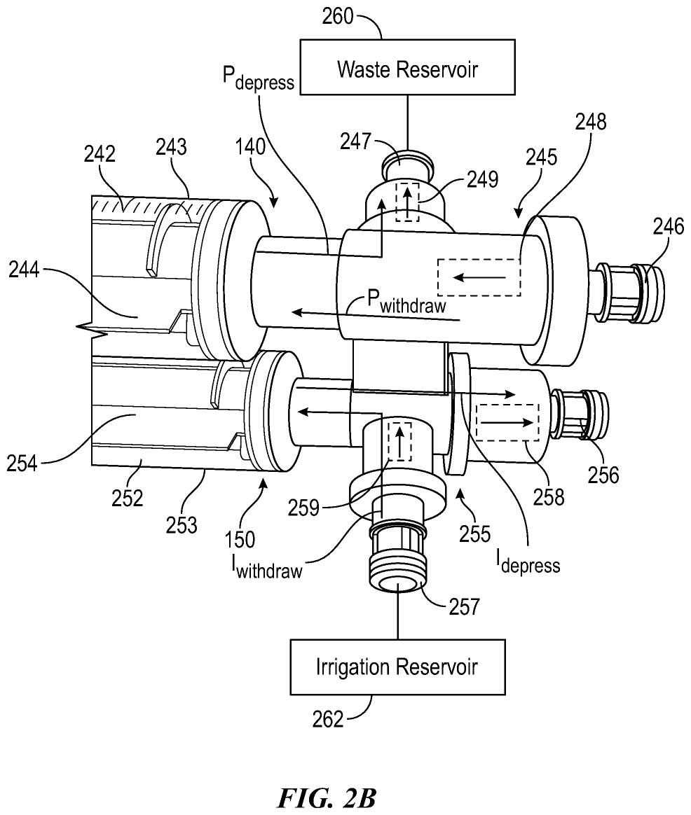

In the illustrated embodiment, the first tubing assembly 120 fluidly couples the inner lumen 111 of the inner elongate member 112 of the catheter assembly 110 to a pressure source assembly 140, such as a syringe and one or more valves as described in detail below with reference to FIGS. 2A and 2B. Similarly, the second tubing assembly 130 fluidly couples the outer lumen 117 of the outer elongate member 114 to an irrigation assembly 150, such as a syringe and one or more valves as described in detail below with reference to FIGS. 2A and 2B. Referring to FIG. 1A, the first and second tubing assemblies 120, 130 (“tubing assemblies 120, 130”) can be generally similar or identical. For example, in the illustrated embodiment the first tubing assembly 120 includes one or more tubing sections 122 (individually labeled as a first tubing section 122a and a second tubing section 122b), at least one fluid control device 124 (e.g., a valve), and at least one connector 126 (e.g., a Toomey tip connector) for fluidly coupling the first tubing assembly 120 to the pressure source assembly 140 and/or other suitable components. Similarly, in the illustrated embodiment the second tubing assembly 130 includes one or more tubing sections 132 (individually labeled as a first tubing section 132a and a second tubing section 132b), at least one fluid control device 134 (e.g., a valve), and at least one connector 136 (e.g., a Toomey tip connector) for fluidly coupling the second tubing assembly 130 to the irrigation assembly 150 and/or other suitable components. Referring to FIGS. 1A and 1B, in some embodiments the fluid control device 124 comprises a stopcock that is fluidly coupled to (i) the inner lumen 111 of the inner elongate member 112 via the second tubing section 122b and (ii) the connector 126 via the first tubing section 122a. Likewise, the fluid control device 134 can comprise a stopcock that is fluidly coupled to (i) the outer lumen 117 of the outer elongate member 114 via the second tubing section 132b and (ii) the connector 136 via the first tubing section 132a. The fluid control devices 124, 134 are externally operable by a user to regulate the flow of fluid therethrough and, specifically, from the inner lumen 111 and to the outer lumen 117, respectively, of the catheter assembly 110 to the pressure source assembly 140 and from the irrigation assembly 150, respectively. In some embodiments, the connectors 126, 136 are quick-release connectors (e.g., quick disconnect fittings) that enable rapid coupling/decoupling of the catheter assembly 110 from the pressure source assembly 140 and/or the irrigation assembly 150.

The system 100 can further include a dilator 108 insertable through the inner lumen 111 of the inner elongate member 112 via the valve 102. The dilator 108 can include a proximal coupling portion 109 configured to be secured to and/or mate to a corresponding portion of the valve 102. In some embodiments, the dilator 108 and/or the valve 102 can be of the type disclosed in U.S. patent application Ser. No. 18/156,944, filed Jan. 19, 2023, and titled “CLOT TREATMENT SYSTEMS WITH DILATOR LOCKING MECHANISMS, AND ASSOCIATED DEVICES AND METHODS,” which is incorporated herein by reference in its entirety.

FIGS. 2A and 2B are a side view and an enlarged partially-schematic side view of the pressure source assembly 140 and the irrigation assembly 150 in accordance with embodiments of the present technology. Referring to FIGS. 2A and 2B, the pressure source assembly 140 includes a pressure source 242, such as a syringe 242 having a barrel 243 and a plunger 244 slidable through the barrel 243, and an aspiration flow control assembly 245 fluidly coupled to the barrel 243. The aspiration flow control assembly 245 can include (i) a first connector 246 (ii) a second connector 247 (obscured in FIG. 2A), (iii) a first one-way valve 248 (shown schematically in FIG. 2B) fluidly coupling the first connector 246 to the barrel 243, and (iv) a second one-way valve 249 (shown schematically in FIG. 2B) fluidly coupling the second connector 247 to the barrel 243 and the first one-way valve 248. Similarly, the irrigation assembly 150 can include a pressure source 252, such as a syringe 252 having a barrel 253 and a plunger 254 slidable through the barrel 253, and an irrigation flow control assembly 255 fluidly coupled to the barrel 253. The irrigation flow control assembly 255 can include (i) a first connector 256, (ii) a second connector 257, (iii) a first one-way valve 258 (shown schematically in FIG. 2B) fluidly coupling the first connector 256 to the barrel 253, and (iv) a second one-way valve 259 (shown schematically in FIG. 2B) fluidly coupling the second connector 257 to the barrel 253 and the first one-way valve 258. In other embodiments, the pressure sources 242, 252 can be other types of pumps or sources of fluid pressure. In some embodiments, the plungers 244, 254 are coupled together via a handle 264 (FIG. 2A) such that the plungers 244, 254 are constrained to move together. In other embodiments, the syringes 242, 252 can be separated such that they are independently operable and/or can have different sizes. In some embodiments, the syringes 242, 252 can have a volume of about 60 cubic centimeters, and can have a large bore coupling of the type described in, for example, U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety.

Referring to FIGS. 1A-2B, the first connector 246 of the pressure source assembly 140 can be connected to the connector 126 of the first tubing assembly 120 to fluidly couple the inner lumen 111 of the catheter assembly 110 to the syringe 242, and the first connector 256 of the irrigation assembly 150 can be connected to the connector 136 of the second tubing assembly 130 to fluidly couple the outer lumen 117 of the catheter assembly 110 to the syringe 252. Referring to FIG. 2B, the second connector 247 of the pressure source assembly 140 can be connected to a waste reservoir 260 (e.g., a waste bag), and the second connector 257 of the irrigation assembly 150 can be connected to an irrigation reservoir 262 (e.g., a saline bag) configured to hold an irrigation fluid. The irrigation fluid can be a sterile fluid having a relatively low viscosity, such as saline.

Arrows P_{withdraw} and P_{depress} in FIG. 2B over the aspiration flow control assembly 245 illustrate the operation of the aspiration flow control assembly 245 when the plunger 244 is withdrawn and depressed, respectively. The first one-way valve 248 of the aspiration flow control assembly 245 can be positioned to, upon pullback (e.g., withdrawal) of the plunger 244, permit fluid flow through the first connector 246 (e.g., from the inner lumen 111) to the barrel 243 of the syringe 242. Concurrently, the second one-way valve 249 of the aspiration flow control assembly 245 inhibits (e.g., blocks) backward fluid flow from the waste reservoir 260 into the barrel 243 of the syringe 242. When the plunger 244 is depressed (e.g., advanced), the second one-way valve 249 can be positioned to permit fluid flow through the second connector 247 (e.g., from the barrel 243) to the waste reservoir 260. Concurrently, the first one-way valve 248 inhibits (e.g., blocks) fluid flow from the barrel 243 into the inner lumen 111.

Similarly, arrows I_{withdraw} and I_{depress} in FIG. 2B over the irrigation flow control assembly 255 illustrate the operation of the irrigation flow control assembly 255 when the plunger 254 is withdrawn and depressed, respectively. The second one-way valve 259 of the irrigation flow control assembly 255 can be positioned to, upon pullback (e.g., withdrawal) of the plunger 254, permit fluid flow through the second connector 257 (e.g., from the irrigation reservoir 262) to the barrel 253 of the syringe 252. Concurrently, the first one-way valve 258 of the irrigation flow control assembly 255 inhibits (e.g., blocks) backward fluid flow from first connector 256 into the barrel 253 of the syringe 252. When the plunger 254 is depressed (e.g., advanced), the first one-way valve 258 can be positioned to permit fluid flow through the first connector 256 (e.g., from the barrel 253) to the outer lumen 117. Concurrently, the second one-way valve 259 inhibits (e.g., blocks) fluid flow from the barrel 253 into the irrigation reservoir 262.

Referring to FIGS. 1A-2B, the catheter assembly 110 can be introduced into a patient through a percutaneous opening (e.g., an opening in the abdomen, an opening in an intercostal space) and advanced such that the distal portion of the catheter assembly 110 is positioned within and/or proximate to a cavity within the body of the patient. The proximal portion 109 of the dilator 108 can be decoupled from the valve 102 and the dilator 108 can be removed from the catheter assembly 110 within the body. Then, the handle 264 can be withdrawn to withdraw the plungers 244, 254 within the barrels 243, 253, respectively. The withdrawal of the plungers 244, 254 simultaneously (i) aspirates the inner lumen 111 of the inner elongate member 112 to aspirate material from within the cavity and (ii) fills (e.g., primes) the syringe 252 with irrigation fluid from the irrigation reservoir 262. Then, the handle 264 can be depressed to depress the plungers 244, 254 within the barrels 243, 253, respectively. The depression of the plungers 244, 254 simultaneously (i) empties the aspirated contents from the cavity from the barrel 243 into the waste reservoir 260 and (ii) forces the irrigation fluid from the barrel 253 into the outer lumen 117 of the outer elongate member 114 and through the apertures 118 into the cavity to irrigate the cavity. Table 1 below illustrates such as an operation of the system 100:

TABLE 1

| Step No. | Aspiration Syringe 242 | Irrigation Syringe 252 |
|---|---|---|
| 1. Pull Syringes | Aspirate Cavity | Prime with Saline |
| 2. Push Syringes | Empty into Waste Bag | Irrigate Cavity |

In some aspects of the present technology, operation of the syringe 242 can aspirate material from the cavity while operation of the syringe 252 can irrigate the cavity to disrupt and/or lower the viscosity of the material therein. Simultaneous operation of the syringes 242 and 252 can ensure constant volume retention within the cavity, such that the aspirated volume is replaced with irrigation volume. In additional aspects of the present technology, the aspiration and irrigation circuits 140, 150, respectively, are independently controlled by the first and second one-way valves 248, 249 of the aspiration flow control assembly 245 and the first and second one-way valves 258, 259 of the irrigation flow control assembly 255, respectively, connected to the syringes 242, 252, respectively. The flow control assemblies can ensure that each of the inner and outer lumens 111, 117 is a one-way path so that the irrigation apertures 118 do not become clogged as, for example, conventional drainage catheter side-holes can when placed in complex collections for aspiration. Moreover, any contents inside the aspiration lumen 111 will not be reintroduced into the cavity during irrigation. That is, the irrigation fluid is introduced via the outer lumen 117 which is separate from the aspiration lumen 111 such that irrigation does not reintroduce any aspirated material into the cavity and aspiration does not clog the fluid pathway for the irrigation fluid.

The syringes 242, 252 can be repeatedly actuated to provide multiple instances of aspiration/irrigation. In some embodiments, the system 100 can be used in a single-session to treat the cavity and entirely or substantially entirely remove the contents thereof, such that the cavity would be effectively drained at the initial treatment and there would be no need to leave a drainage catheter behind. In other embodiments, after treating the cavity with the system 100, a drainage catheter can be inserted into the cavity after aspiration and irrigation treatment. The system 100 can be left in the patient to function as a drainage system for a partial amount or the full duration of drainage, and/or a separate drainage catheter (e.g., having a smaller size) can be inserted into the patient and the system 100 removed for further drainage. That is, the system 100 can be used for initial debridement, drainage, and flushing of the contents of the cavity, and a standard commercial drain could then be inserted upon the removal of the system 100, within the same procedure, to allow for any remaining collection to be drained over the subsequent days. In some aspects of the present technology, use of the system 100 can eliminate the need for off-label mechanical devices and pharmacological agents for drainage, thus improving patient outcomes and reducing duration of drainage.

In additional aspects of the present technology, the catheter assembly 110 can be optimized to maximize drainage (e.g., aspiration) flow. For example, the catheter assembly 110 can be optimized in view of Poiseuille's law:

$$Q = \frac{π * Δ P * {R}^{4}}{8 * η * L}$$

Where Q is the flow rate through a tube, ΔP is the pressure differential between the tube inlet and outlet, R is the radius of the tube (shown in FIG. 1B), η is the fluid viscosity, and L is the tube length. Assuming the pressure difference ΔP and length L of the catheter assembly 110 are constant between an aspiration catheter and a standard drainage catheter, the present technology can reduce the collection viscosity η while maintaining a large radius R (FIG. 1B) of the inner lumen 111 (e.g., the drainage lumen). For example, the introduction of the irrigation fluid via the outer lumen 117 can reduce the collection viscosity η while the coaxial arrangement of the inner and outer lumens 111, 117 can maximize the radius R of the inner lumen 111. In contrast, some conventional double-lumen drains (e.g., sumps) have been developed to promote drainage by providing a venting lumen. However, the introduction of such a secondary lumen compromises the radius of the collection lumen and thus decreases flow. Accordingly, the system 100 can provide an optimal drainage catheter solution that (i) maximizes the drainage lumen (e.g., the inner lumen 111) of the catheter assembly 110, (ii) reduces collection viscosity via irrigation through the outer lumen 117, and/or (iii) breaks apart loculations and debris via the vigorous irrigation through the outer lumen 117.

Accordingly, the system 100 can be designed to maximize flow of material by utilizing Poiseuille's law as defined above. In some embodiments, the pressure differential is maximized by using a 60 cc syringe 242 which can create a vacuum of −25.5 inHg when fully evacuated. The radius of the inner elongate member 112 is maximized by maintaining a single lumen at the same diameter from the distal tip to the syringe 242 by utilizing a large bore side port tubing (e.g., within the first tubing assembly 120) and a large bore syringe 242. Fluid viscosity can be lowered via the irrigation process which can dilute the cavity contents with an irrigation fluid of low viscosity. The length of the system 100 can be minimized by maintaining as short a distance as possible between the tip of the catheter assembly 110 and the vacuum source/syringe 242. In contrast, current drains can use excess tubing length to connect to gravity collection bags, wall suction, or suction bulbs, which decreases the efficiency of the drain.

In contrast to the present technology, a typical dual-lumen catheter (either extruded or as part of a braided catheter) will face challenges in maintaining a reduced overall profile. Some common designs include a second lumen within or adjacent to the main aspiration lumen. These designs suffer from an excessive outer diameter and a single lumen for irrigation which lies in the same plane as the aspiration lumen. In some aspects of the present technology, the two coaxial lumens 111, 117 provides several advantages for this application: (i) a continuous circular inner lumen 111 to maximize inlet area to the aspiration lumen 111, and (ii) a concentric reservoir for circumferential (3-dimensional) irrigation via the outer lumen 117. In some aspects of the present technology, circumferential irrigation is important not only to reduce viscosity of the collection, but also to vigorously agitate the local region, potentially disbanding loculations and displacing adherent material. The 3-dimensional pattern of the irrigation fluid distributed via the apertures 118 is helpful in providing more distributed, targeted irrigation that is not limited to a single plane as it would be in a single-lumen configuration. For example, a small single lumen (˜1 French −4 French) for infusion may only achieve local viscosity reduction without sufficient disruption of the collection.

In other embodiments the pressure source assembly 140 and/or the irrigation assembly 150 can be configured to provide more control and/or operated in different manners. For example, the aspiration syringe 242 and the irrigation syringe 252 can be independently operated or have separately pre-determined volumes for each stroke (e.g., by omitting the handle 264). In some embodiments, the fluid control device 124 can be closed during withdrawal of the plunger 244 such that a vacuum is generated (e.g., pre-charged) within the barrel 243 of the syringe 242. The fluid control device 124 can subsequently be opened to apply the vacuum to the inner lumen 111 and generate a suction/aspiration pulse through the inner lumen 111. Moreover, although the first and second one-way valves 248, 249 are shown to be within the aspiration flow control assembly 245 and the first and second one-way valves 258, 259 are shown to be within the irrigation flow control assembly 255, in other embodiments any or all of the one-way valves 248, 249, 258, 259 can be incorporated directly into the catheter assembly 110 to, for example, avoid any confusion or mixing of the aspiration and irrigation circuits.

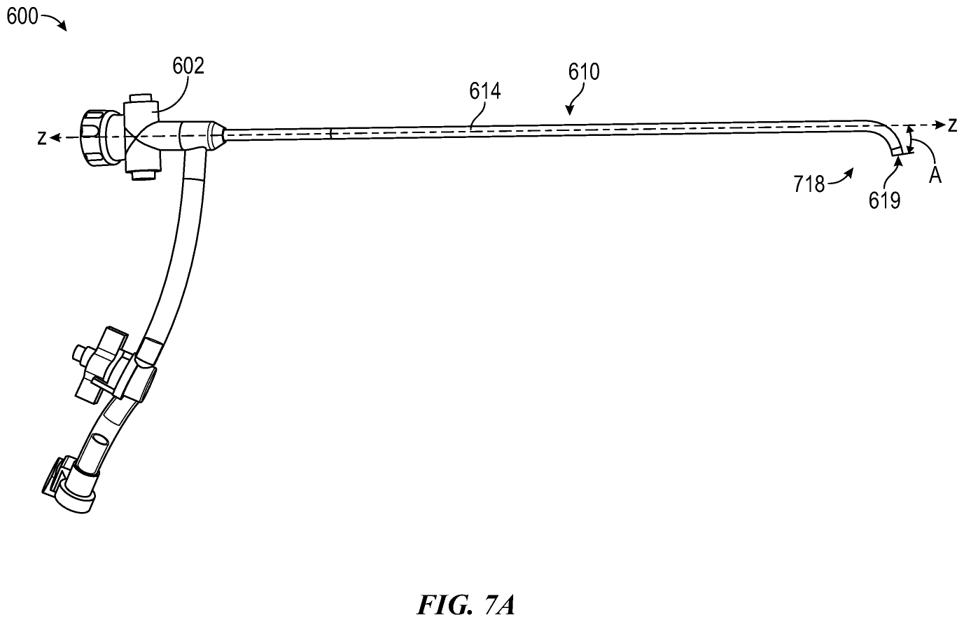

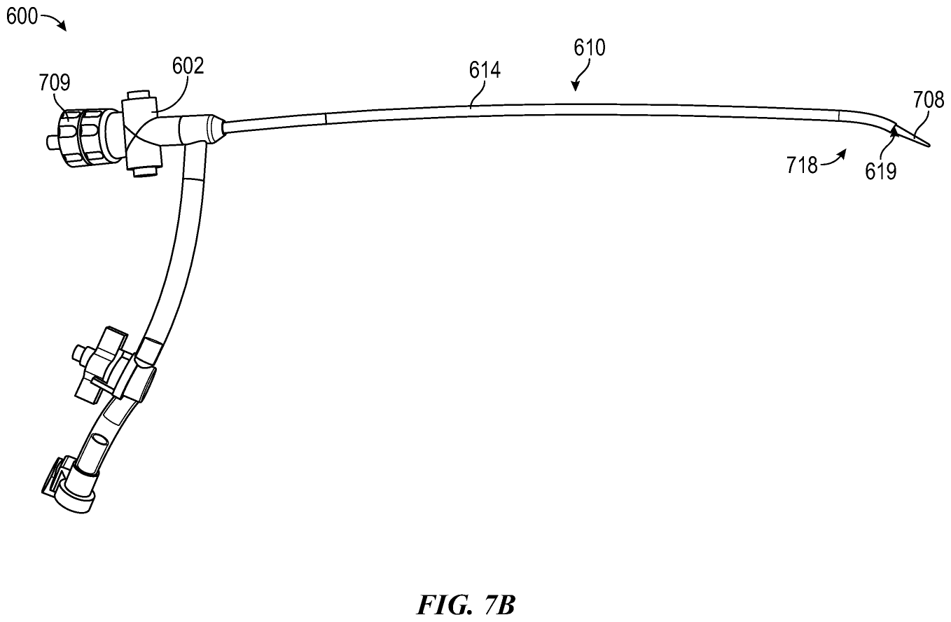

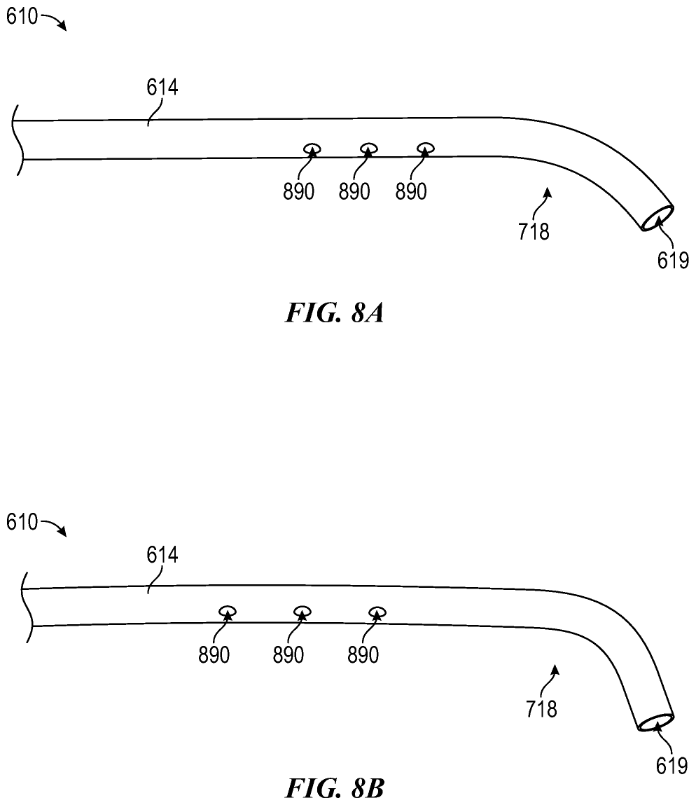

Referring to FIGS. 1A-1C, the system 100 can have various other configurations. For example, (i) the dilator 108 can be long-tipped or short-tipped for insertion of the system 100 into the patient, (ii) the catheter assembly 110 can include a balloon-tip for localized irrigation and retention within the cavity, and/or (iii) the distal portion of the catheter assembly 110 can have various curves (pig-tail, J-hook, etc.; e.g., as shown in FIGS. 7A-8B). Moreover, in some embodiments the outer lumen 117 can be omitted and the catheter assembly 110 can include/define only the inner lumen 111. Such embodiments can provide a simplified design that maintains the benefits of large-bore aspiration/drainage without the ability to irrigate through a separate lumen. The large-bore lumen could still be used for irrigation, if needed. Further, in some embodiments the outer elongate member 114 can define multiple irrigation lumens extending between the second hub 106 and, for example, corresponding ones or multiples of the apertures 118. The individual irrigation lumens can be placed around the inner aspiration lumen 111 and can be formed via a multi-lumen extrusion process, a tri-axial coiling/braiding process, and/or another suitable processes. The individual irrigation lumens can be circular or have other cross-sectional shapes.

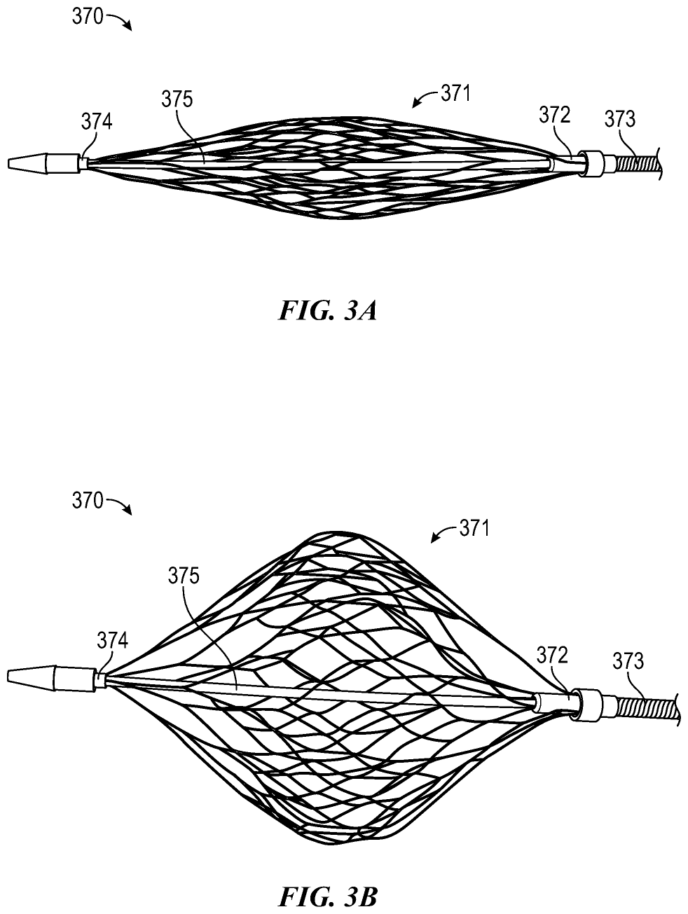

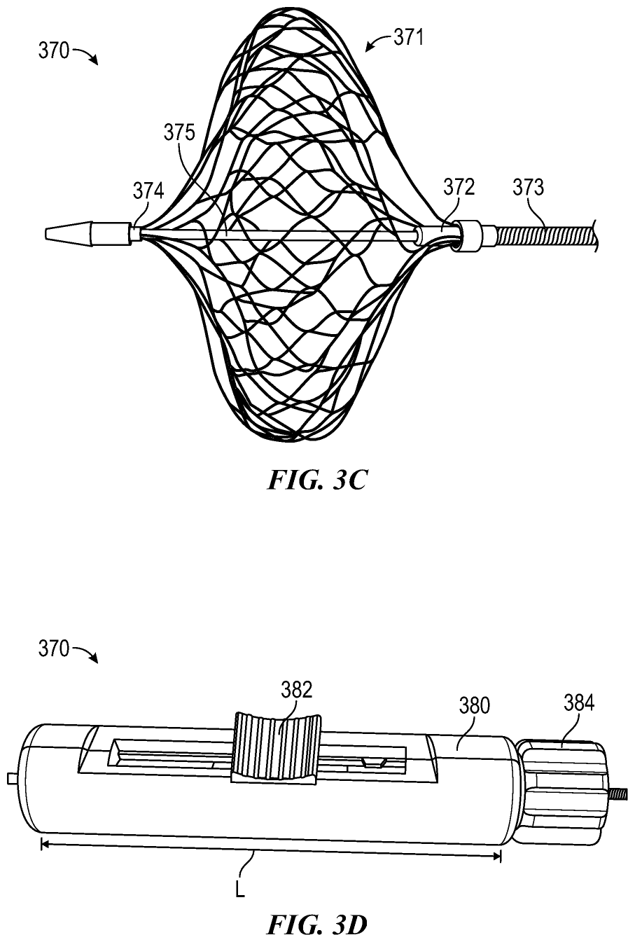

Referring to FIGS. 1A-1C, in some embodiments if disruption of the contents of the cavity by irrigation is inadequate (e.g., where the contents are too viscous or large for aspiration after irrigation), a mechanical tool or element can be deployed within the cavity through the inner lumen 111 of the catheter assembly 110. In some embodiments, the mechanical element is similar to those used in thrombectomy procedures. The mechanical element can have a size and shape that can be controlled to form a desired geometry and manipulated within the cavity to aid with subsequent aspiration and drainage. FIGS. 3A-3C, for example, are enlarged side views of a mechanical disruptor assembly 370 in a compressed position, a partially-expanded position, and an expanded position, respectively, that is configured to be advanced through the catheter assembly 110 to within a cavity to mechanically disrupt material therein in accordance with embodiments of the present technology. Referring to FIGS. 3A-3C, the mechanical disruptor assembly 370 includes a disruptor element 371 comprising a plurality of interconnected struts and having a proximal portion 372 secured to a first elongate shaft 373 and a distal portion 374 secured to a second elongate shaft 375. The second elongate shaft 375 is slidably disposed within the first elongate shaft 373. One or more of the struts can have a sharpened cutting edge and/or one or more of the struts can have an atraumatic edge.

The disruptor element 371 can be made of nitinol braid, tubing, stainless steel, and/or any other biocompatible material. In some embodiments, the mechanical disruptor assembly 370 can include several features generally similar or identical to those of the clot treatment devices described in detail in U.S. patent application Ser. No. 17/072,909, filed Oct. 16, 2020, and titled “SYSTEMS, DEVICES, AND METHODS FOR TREATING VASCULAR OCCLUSIONS,” which is incorporated herein by reference in its entirety.

FIG. 3D is a side view of a handle 380 of the mechanical disruptor assembly 370 in accordance with embodiments of the present technology. Referring to FIGS. 3A-3D, the first and second elongate shafts 373, 375 (e.g., proximal portions thereof) can be operably coupled to the handle 380. The handle 380 can include a first actuator 382 coupled to one of the first elongate shaft 373 or the second elongate shaft 375 and can be actuatable to translate the first and second elongate shafts 373, 375 relative to one another. For example, the first actuator 382 can be coupled to the second elongate shaft 375 such that (i) movement of the first actuator 382 in the proximal direction along an axis L retracts the second elongate shaft 375 proximally relative to the first elongate shaft 373 and (ii) movement of the first actuator 382 in the distal direction along the axis L advances the second elongate shaft 375 distally relative to the first elongate shaft 373. Movement of the first actuator 382 can move the disruptor element 371 between the compressed position, the partially-expanded position, and the expanded position. For example, when the disruptor element is in the compressed position (FIG. 3A), the first actuator 382 can be slid (e.g., proximally) to move the second elongate shaft 375 proximally to move the distal portion 374 of the disruptor element 371 proximally toward the proximal portion 372 of the disruptor element 371 to radially expand the disruptor element to the partially-expanded position (FIG. 3B) and, by further actuation, to the expanded position (FIG. 3C). In other embodiments, the disruptor element 371 can be configured to passively expand (e.g., self-expand) within the cavity in addition to or alternatively to actively via, for example, the handle 380.

In other embodiments, the actuator 382 can have one or more locking positions within the handle 380 to determine a diameter of the disruptor element 371. Accordingly, the disruptor element can have a controllable diameter which can be gradually increased and manipulated to tear apart loculations and/or other material within a body cavity bit by bit until the diameter of the disruptor element 371 reaches the wall of the cavity. In some embodiments, the handle 380 further includes a second actuator 384 (e.g., a rotatable knob) operably coupled to the first and/or second elongate shafts 373, 375. The second actuator 384 can be actuated (e.g., rotated) to rotate the disruptor element 371 to, for example, further break apart material within the cavity, such as material adhered to the wall of the cavity. The mechanical disruptor assembly 370 can be translated laterally by a user if needed (e.g., via movement of the handle 380) and can be delivered through the inner lumen 111 of the catheter assembly 110 via a guidewire or without a guidewire.

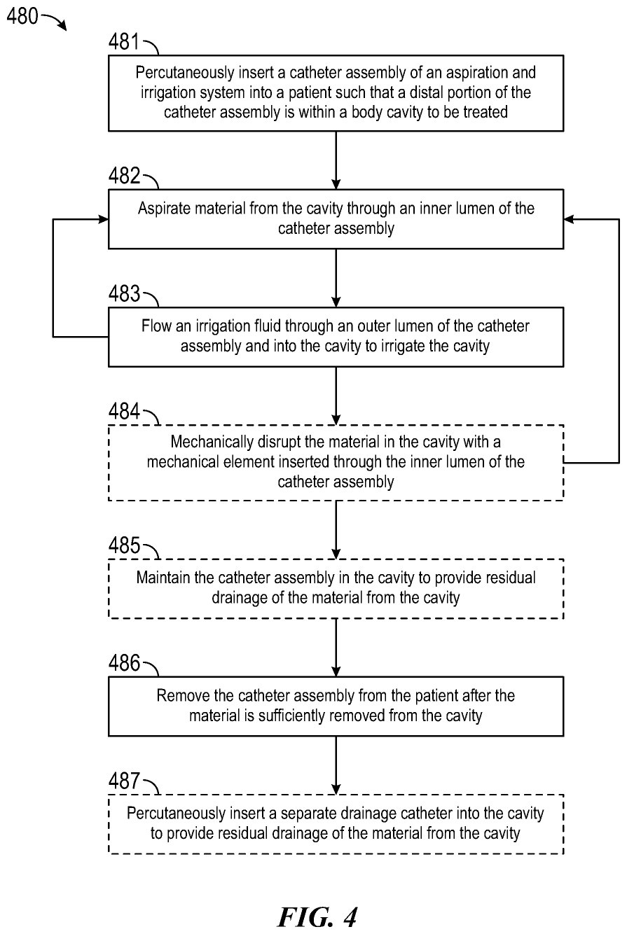

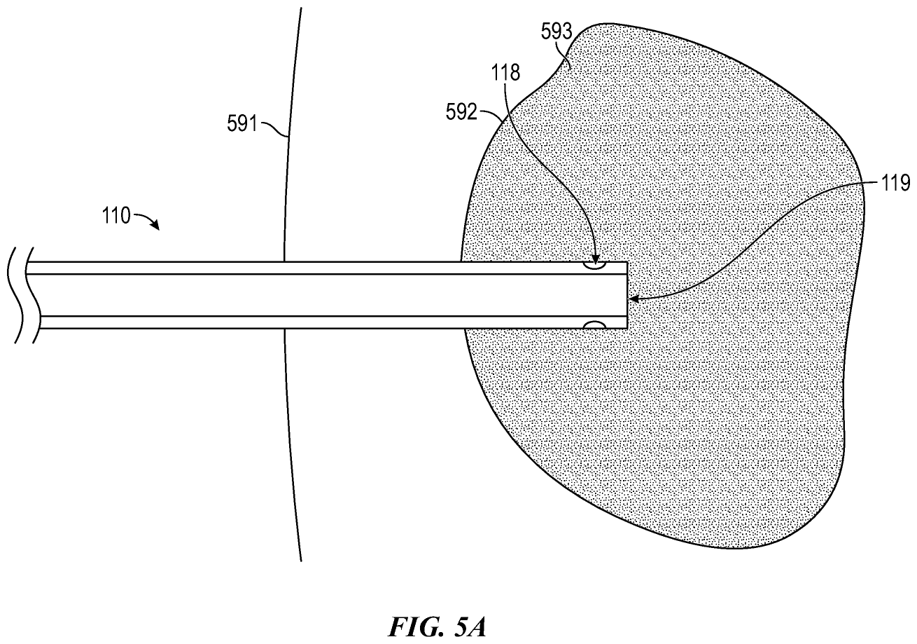

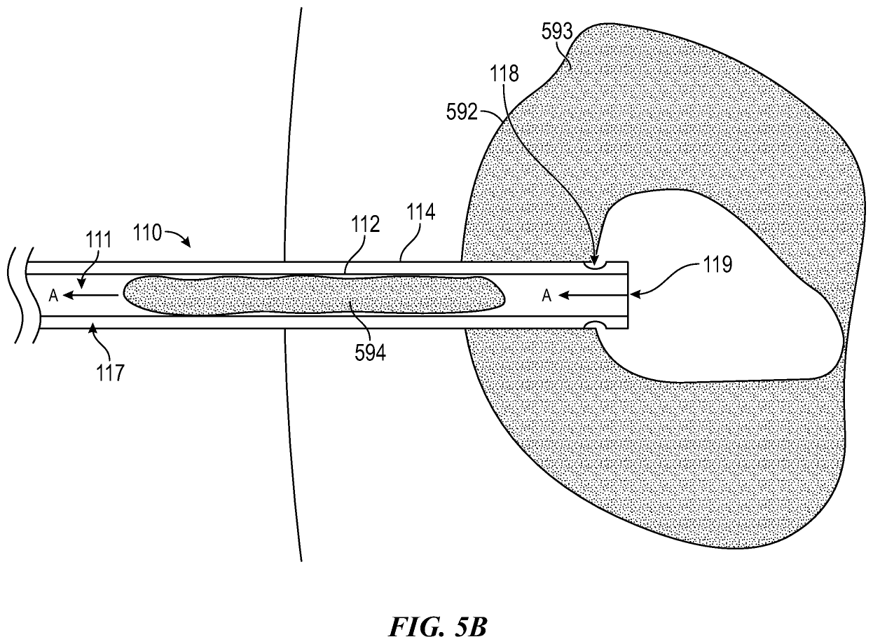

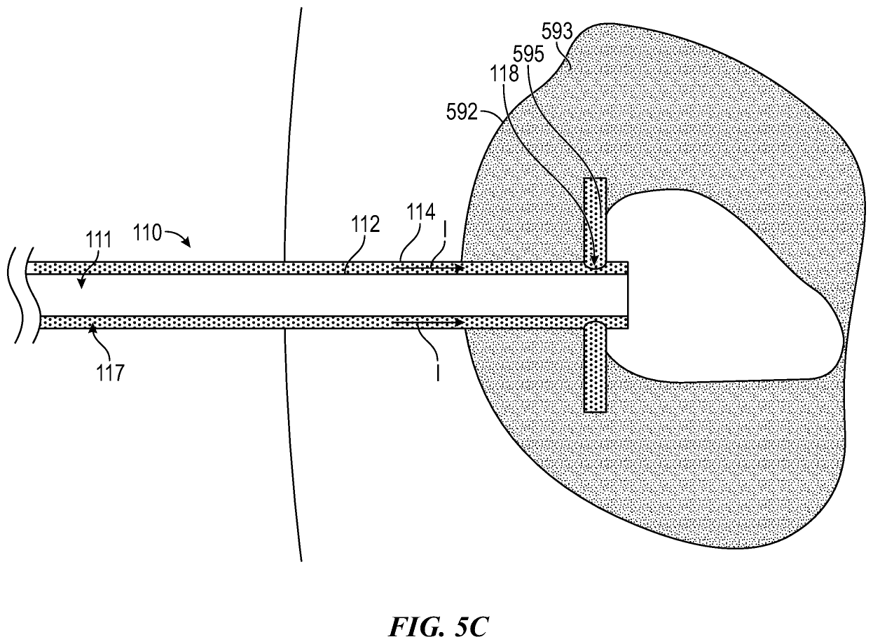

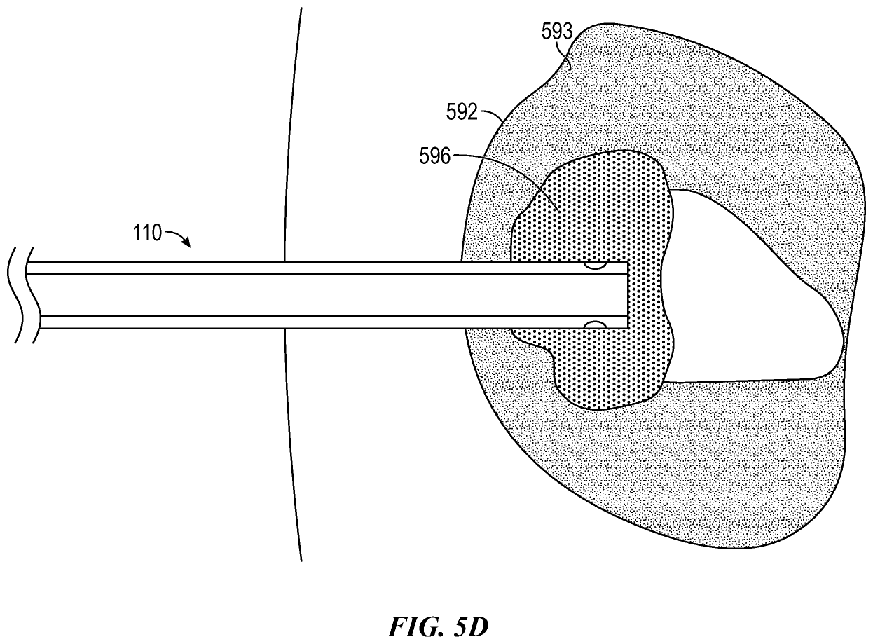

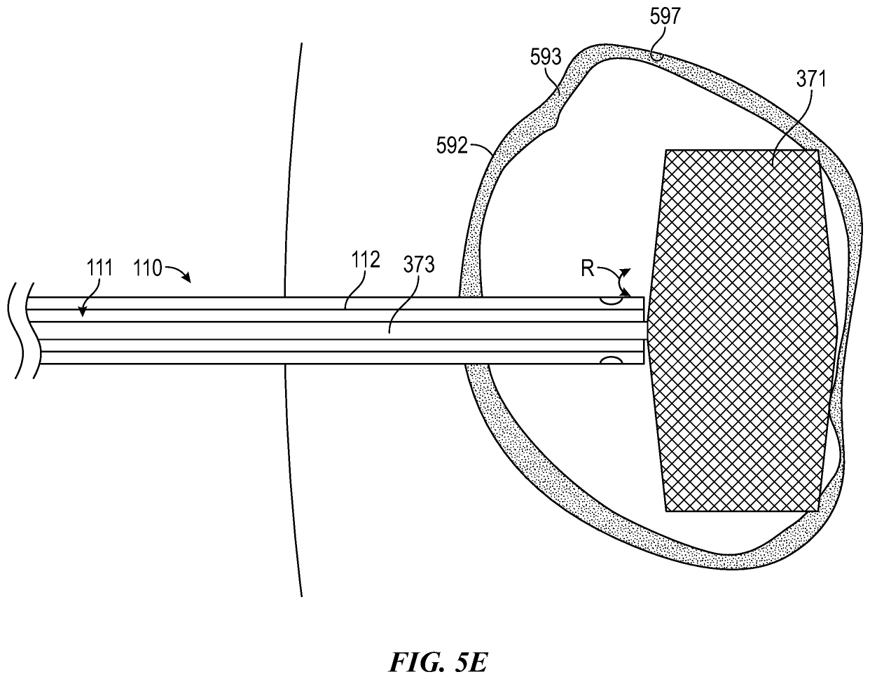

FIG. 4 is a flow diagram of a process or method 480 for treating a body cavity of a patient using the system 100 in accordance with embodiments of the present technology. The body cavity can be an abdominal abscess, an empyema, a (e.g., complicated) pleural effusion, and/or another cavity containing unwanted material/contents. Although some features of the method 480 are described in the context of the embodiments shown in FIGS. 1A-3D for illustration, one skilled in the art will readily understand that the method 480 can be carried out using other suitable systems and/or devices described herein. FIGS. 5A-5E are side cross-sectional views of a distal portion of the catheter assembly 110 during different stages of the method 480 in accordance with embodiments of the present technology.

At block 481, the method 480 can include percutaneously inserting the catheter assembly 110 of the system 100 into the patient such that a distal portion of the catheter assembly 110 is positioned within the cavity to be treated. For example, FIG. 5A shows the catheter assembly 110 interested through skin 591 of a patient and into a body cavity 592 including material or contents 593 positioned therein such that the distal opening 119 and the apertures 118 are positioned within the cavity 592. The material 593 can comprise pus that may be thick and viscous (e.g., purulent fluid), necrotic debris, blood clots, enteral content, loculations, and/or the like. The material 593 can substantially fill the cavity 592 as shown in FIG. 5A, or can partially fill the cavity 592. In some embodiments, the catheter assembly 110 is inserted into the abdomen or into an intercostal space of the patient proximate to the cavity 592 via, for example, the Seldinger technique, Trocar technique, and/or another catheter insertion technique. In some aspects of the present technology, the catheter assembly 110 can have a relatively short length M (FIG. 1A) because the catheter assembly 110 is designed to be inserted into the skin 591 of the patient proximate to the cavity 592.

At block 482, the method 480 can include aspirating material from the cavity through the inner lumen 111 of the catheter assembly 110. For example, as described in detail above with reference to FIGS. 2A and 2B, the syringe 242 can be actuated (e.g., the plunger 244 withdrawn) to aspirate the inner lumen 111. During aspiration, the aspiration flow control assembly 245 is configured to permit fluid flow from the inner lumen 111 to the syringe 242 while simultaneously blocking fluid flow from the waste reservoir 260 to the syringe 242. FIG. 5B shows the catheter assembly 110 during aspiration as an aspiration force indicated by arrows A pulls a portion 594 of the material 593 proximally into and through the inner lumen 111 of the inner elongate member 112 from the distal opening 119. The portion 594 of the material 593 can be pulled entirely through the inner lumen 111, through the first tubing assembly 120, through the aspiration and flow control assembly 245, and into the barrel 243 of the syringe 242 where it is collected. In some aspects of the present technology, the material 593 does not or substantially does not enter the outer lumen 117 of the outer elongate member 114 during aspiration because (i) the apertures 118 lay in a different plane than the distal opening 119 (e.g., in orthogonal planes) and (ii) only the inner lumen 111 is aspirated such that clogging of the apertures 118 is inhibited or even prevented.

At block 483, the method 480 can include flowing an irrigation fluid from the irrigation reservoir 262 through the outer lumen 117 of the catheter assembly 110 and out of the apertures 118 into the cavity to irrigate the cavity. For example, as described in detail above with reference to FIGS. 2A and 2B, the syringe 252 can be primed with the irrigation fluid by withdrawing the plunger 254 (e.g., at the same time the plunger 244 of the syringe 242 is withdrawn to aspirate the inner lumen 111) and then depressed to drive the irrigation fluid through the irrigation flow control assembly 255 into the outer lumen 117. As the plunger 254 is depressed, the irrigation flow control assembly 255 permits the irrigation fluid to flow into the outer lumen 117 while blocking the irrigation fluid from flowing into the irrigation reservoir 262. FIG. 5C shows the catheter assembly 110 during irrigation as an irrigation fluid 595 is driven through the outer lumen 117 as indicated by arrows I and through the apertures 118 into the cavity 592. In some aspects of the present technology, the irrigation fluid 595 does not flow through the inner lumen 111 such that any portion of the material 593 remaining within the inner lumen 111 after aspiration is not reintroduced into the cavity 592. The irrigation fluid 595 can exit the apertures 118 as a jet that mechanically disrupts (e.g., breaks apart) the material 593 within the cavity 592. As described in detail above, the position, size, shape, and/or orientation of the apertures 118 can be selected to provide a desired jet pattern for disrupting the material 593. The irrigation fluid 595 can also have a lower viscosity than the material 593 within the cavity 592 such that, after irrigation, a mixture of the irrigation fluid 595 and the material 593 within the cavity 592 has a lower viscosity that can be more easily aspirated in a subsequent aspiration operation. For example, FIG. 5D shows the catheter assembly 110 after irrigation when a resulting mixed portion 596 of the material 593 and the irrigation fluid 595 (FIG. 5C) has a lower viscosity than the material 593.

After irrigation at block 483, the method 480 can return to block 482 to again aspirate the cavity before again proceeding to block 483 to irrigate the cavity. Aspiration and irrigation can be performed as many times as necessary to sufficiently remove the material from the cavity. Optionally, at block 484, the method 480 can include mechanically disrupting (e.g., debriding) the material in the cavity with a mechanical element, such as the disruptor element 371, that is inserted through the inner lumen 111. FIG. 5E, for example, shows the catheter assembly 110 after insertion of the disruptor element 371 (shown schematically in FIG. 5E) into the cavity 592 through the inner lumen 111 of the inner elongate member 112, and after expansion of the disruptor element 371. The disruptor element 371 can be rotated as indicated by arrow R (e.g., about the first elongate shaft 373) within the cavity 592 and/or translated (e.g., proximally and/or distally) within the cavity 592 to mechanically disrupt any of the material 593 remaining after aspiration and irrigation (blocks 482 and 483). In some embodiments, the disruptor element 371 can be actuated to mechanically disrupt some or all of the material 593 that remains adhered to a wall 597 of the cavity 592 after aspiration and irrigation.

In other embodiments, the material in the cavity can be mechanically disrupted before aspiration and irrigation (blocks 482 and 483). That is, for example, the disruptor element 371 can be inserted through the inner lumen 111 and into the cavity 592 and rotated and/or translated within the cavity 592 to initially mechanically disrupt and/or break apart the material 593. In some aspects of the present technology, mechanically disrupting the material 593 before aspiration and irrigation can make the aspiration and irrigation more effective.

At block 485, the method 480 can include maintaining the catheter assembly 110 in the cavity to provide residual drainage of material from the cavity. The catheter assembly 110 can be maintained within the cavity for hours, days, or weeks to provide residual drainage.

At block 486, the method 480 can include removing the catheter assembly 110 from the patient after the material is sufficiently removed from the cavity. At block 487, the method 480 can optionally include percutaneously inserting a separate drainage catheter into the cavity to provide (further) residual drainage. In some aspects of the present technology, the drainage catheter can have a smaller size (e.g., for improved patient comfort), can be configured for connection to existing drainage waste bags, and/or can be of a type familiar to an operator (e.g., hospital staff). In such embodiments, the system 100 can be used for initial debridement, drainage, and flushing of the contents of the cavity (blocks 482-484), and the separate drainage catheter can be a standard commercial drain that is inserted upon the removal of the system 100 (block 486), within the same procedure, to allow for any remaining collection to be drained over the subsequent days.

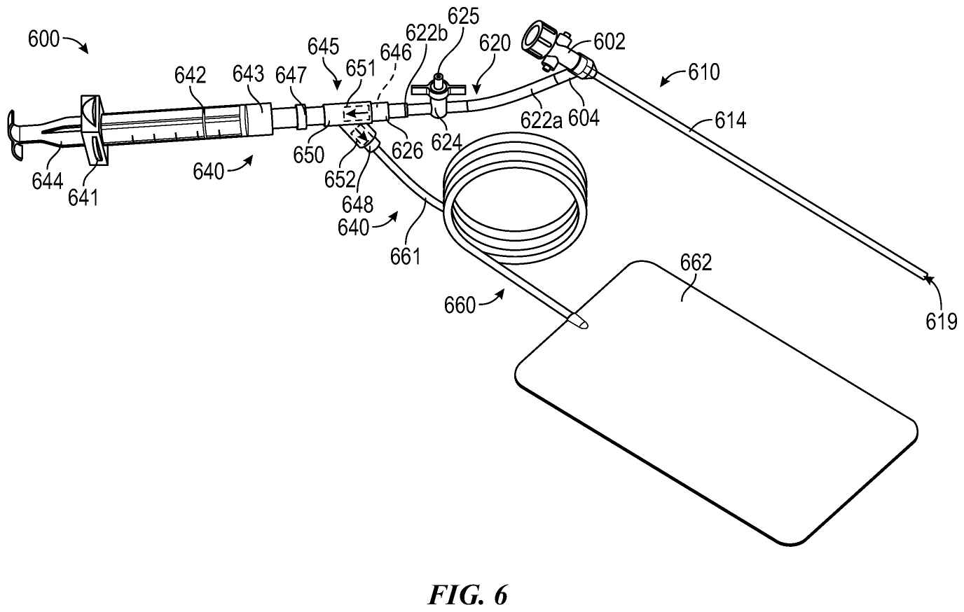

FIG. 6 is a side view of an aspiration and irrigation system 600 (“system 600”) in accordance with additional embodiments of the present technology. The system 600 can include some features that are at least generally similar in structure and function, or identical in structure and function, to the corresponding features of the system 100 described in detail above with reference to FIGS. 1A-5E, and can operate in a generally similar or identical manner to the system 100. For example, in the illustrated embodiment the system 600 includes a catheter assembly 610 fluidly coupled to (i) a valve 602 and (ii) a tubing assembly 620 via a hub or side port 604.

In the illustrated embodiment, the catheter assembly 610 defines a single lumen that can be used to provide both aspiration and irrigation. That is, the catheter assembly 610 can include only a single elongate member 614 (e.g., a sheath, a catheter, a shaft) extending distally from the valve 602 and the hub 604 and defining the lumen. The lumen can terminate at a distal opening 619 (e.g., an aspiration and irrigation opening). The elongate member 614 can have a size of between about 6-30 French, such as a size of 6 French, 8 French, 12 French, 16 French, 20 French, 24 French, 26 French, or 30 French. The lumen of the elongate member 614 is fluidly coupled to (i) the valve 602 and (ii) the tubing assembly 620 via the hub 604. The valve 602 can be an actuated access valve as described in detail above with reference to FIGS. 1A-1C that is configured to maintain fluid control during a body cavity treatment procedure by inhibiting or preventing fluid flow in the proximal direction through the valve 602 as various components such as delivery sheaths, pull members, guidewires, interventional devices, mechanical disruptor assemblies, other aspiration catheters, and so on, are inserted through the valve 602 to be delivered through the elongate member 614 to a treatment site in a body cavity. In some embodiments, the lumen of the elongate member 614 has a constant or substantially constant diameter extending from the distal opening 619 to the valve 602. That is, for example, the elongate member 614 does not have a tapered or reduced diameter portion at the distal end portion thereof or elsewhere along the length of the elongate member 614. In some aspects of the present technology, this can maximize aspiration flow rates throughout the lumen of the elongate member 614.

The tubing assembly 620 can fluidly couple the lumen of the elongate member 614 to a pressure source assembly 640 and/or an irrigation assembly (not shown). For example, in the illustrated embodiment the tubing assembly 620 includes one or more tubing sections 622 (individually labeled as a first tubing section 622a and a second tubing section 622b), at least one fluid control device 624 (e.g., a valve, a stopcock), and at least one connector 626 (e.g., a Toomey tip connector, a quick-release connector) for fluidly coupling the tubing assembly 620 to the pressure source assembly 640, the irrigation assembly, and/or other suitable components.

The pressure source assembly 640 can comprise a pressure source 642, such as a syringe 642 having a barrel 643 and a plunger 644 slidable through the barrel 643, and an aspiration flow control assembly 645 fluidly coupled to the barrel 643 of the syringe 642. In some embodiments, the syringe 642 includes a lock mechanism 641 configured to selectively lock the plunger 644 relative to the barrel 643 (e.g., in a withdrawn position). Accordingly, the syringe 642 can be an automatically-locking syringe and can include some features that are at least generally similar in structure and function, or identical in structure and function, to those of the automatically-locking syringes discloses in U.S. patent application Ser. No. 17/396,426, filed Aug. 6, 2021, and titled “AUTOMATICALLY-LOCKING VACUUM SYRINGES, AND ASSOCIATED SYSTEMS AND METHODS,” which is incorporated herein by reference in its entirety.

The aspiration flow control assembly 645 can include a body 650 having (i) a first connector 646 (partially obscured in FIG. 6) configured to be coupled to the connector 626 of the tubing assembly 620, (ii) a second connector 647 configured to be coupled to the syringe 642 (e.g., to a tip thereof), and (iii) a third connector 648 configured to be coupled to a waste reservoir 660 and, more particularly, to at least one tube 661 of the waste reservoir 660 fluidly coupled to a waste collection bag 662 or other fluid reservoir of the waste reservoir 660. The body 650 can define one or more lumens that fluidly couple the first through third connector 646-648. In the illustrated embodiment, the aspiration flow control assembly 645 further includes a first one-way valve 651 (shown schematically in FIG. 6) in the flow path between the first connector 646 and the second connector 647, and a second one-way valve 652 (shown schematically in FIG. 6) in the flow path between the second connector 647 and the third connector 648. The first one-way valve 651 is positioned to permit fluid flow from the tubing assembly 620 and the lumen of the elongate member 614 through the aspiration flow control assembly 645 to the barrel 643 of the syringe 642 (e.g., from the first connector 646 to and through the second connector 647), while inhibiting fluid flow from the syringe 642 to the tubing assembly 620 (e.g., from the second connector 647 to and through the first connector 646). The second one-way valve 652 is positioned to permit fluid flow from the barrel 643 of the syringe 642 through the aspiration flow control assembly 645 to the waste reservoir 660 (e.g., from the second connector 647 to and through the third connector 648), while inhibiting fluid flow from the waste reservoir 660 to the syringe 642 (e.g., from the third connector 648 to and through the second connector 647).

Accordingly, withdrawal of the plunger 644 through the barrel 643 generates negative pressure in the barrel 643 that draws fluid through the distal opening 619 and the lumen of the elongate member 614, through the tubing assembly 620, through the aspiration flow control assembly 645 (e.g., through the first connector 646, the first one-way valve 651, and the second connector 647), and into the barrel 643 of the syringe 642. During withdrawal of the plunger 644, the second one-way valve 652 inhibits (e.g., blocks) backward fluid flow from the waste reservoir 660 into the barrel 643 of the syringe 642. Conversely, depression of the plunger 644 through the barrel 643 generates positive pressure in the barrel 643 that forces fluid from the barrel 643 through the aspiration flow control assembly 645 (e.g., through the second connector 647, the second one-way valve 652, and the third connector 648) and into the waste reservoir 660 (e.g., through the tube 661 and into the collection bag 662). During depression of the plunger 644, the first one-way valve 651 inhibits (e.g., blocks) fluid flow from the barrel 643 into the tubing assembly 620 and the lumen of the elongate member 614.

In some embodiments, the pressure source assembly 640 can be decoupled from the connector 626 of the tubing assembly 620 (e.g., with the fluid control device 624 in a closed position) and the irrigation assembly can be coupled to the connector 626 to fluidly couple the irrigation assembly to the lumen of the elongate member 614. In some embodiments, the irrigation assembly can include a syringe or other pressure source and a reservoir of irrigation fluid (e.g., as described in detail above with reference to FIGS. 2A and 2B). The pressure source can be activated (e.g., a plunger of the syringe depressed) to drive the irrigation fluid through the tubing assembly 620, through the lumen of the elongate member 614, and out of the distal opening 619 of the elongate member 614. In some embodiments, the irrigation assembly can alternatively or additionally be coupled to a port 625 of the fluid control device 624 that provides a fluid connection to the tubing assembly 620 and the lumen of the elongate member 614. In such embodiments, the pressure source of the irrigation assembly can be activated to drive the irrigation fluid through the port 625, through the tubing assembly 620, through the lumen of the elongate member 614, and out of the distal opening 619 of the elongate member 614.

In operation, the catheter assembly 610 can be introduced into a patient through a percutaneous opening (e.g., an opening in the abdomen, an opening in an intercostal space) and advanced such that a distal portion of the catheter assembly 610 (e.g., the distal opening 619 of the elongate member 614) is positioned within and/or proximate to a cavity within the body of the patient. The pressure source assembly 640 can be coupled to the tubing assembly 620 and the fluid control device 624 can be opened to fluidly connect the pressure source assembly 620 to the lumen of the elongate member 614. The plunger 644 of the syringe 642 can then be withdrawn to draw fluid from the lumen of the elongate member 614 to aspirate material from within the cavity into the barrel 643. The plunger 644 can then be depressed to drive the material from the barrel 643 into the collection bag 662. In some embodiments, the plunger 644 can be repeatedly withdrawn and subsequently depressed (e.g., pumped) to aspirate the cavity and expel the aspirated material into the collection bag 662. In some aspects of the present technology, the collection bag 662 provides a sealed receptacle for the aspirated material that allows for a cleaner procedure by reducing mess, foul odor, exposure to infected material, and/or the like. In some embodiments, the fluid control device 624 can be closed during withdrawal of the plunger 644 such that a vacuum is generated (e.g., pre-charged) within the barrel 643 of the syringe 642. The fluid control device 624 can subsequently be opened to apply the vacuum to the lumen of the elongate member 614 and generate a suction/aspiration pulse through the lumen to aspirate the material within the cavity.

At any point during the procedure, the fluid control device 624 can be closed and the pressure source assembly 640 decoupled from the tubing assembly 620. The irrigation assembly can then be coupled to the tubing assembly 620, the fluid control device 624 opened, and the irrigation assembly activated to drive irrigation fluid into the lumen of the elongate member 614 and out of the distal opening 619 into the cavity of the patient. Multiple irrigation passes/cycles can be performed. Alternatively or additionally, the pressure source assembly 640 can remain coupled to the tubing assembly 620 and the irrigation assembly can be coupled to the port 625 to provide irrigation through the lumen of the elongate member 614. Irrigation and aspiration can be provided in any order and repeated as needed, such as aspiration first then irrigation, irrigation first then aspiration, one or more cycles of aspiration followed by one or more cycles of irrigation, one or more cycles of irrigation followed by one or more cycles of aspiration, and so on.

In some embodiments, a distal portion of the elongate member 614 can be curved to facilitate placement and positioning within a cavity of a patient. FIG. 7A, for example, is a side view of the catheter assembly 610 in accordance with additional embodiments of the present technology. In the illustrated embodiment, the elongate member 614 has a distal curved portion 718 (e.g., a distal curved end portion, a distal curved region, a distal curved end portion, a curved distal tip, and/or the like) configured (e.g., heat set) to deflect away from a longitudinal axis Z of the catheter assembly 610. In the illustrated embodiment, the distal curved portion 718 has a generally curved shape and can bend away from the longitudinal axis Z by a bend angle A of between about 30-90 degrees (e.g., about 80 degrees), between about 165-195 degrees (e.g., about 180 degrees) or greater than 195 degrees (e.g., between about 250-290 degrees, about 270 degrees). Accordingly, the distal curved portion 718 can have a curvature ranging from a full pigtail to a minor angle. The curvature of the distal curved portion 718 offsets the distal opening 619 from the longitudinal axis Z. During a procedure to treat a cavity of a patient, the elongate member 614 can be torqued (e.g., rotated) to control the position of the distal opening 619 within the cavity to provide directed aspiration and/or irrigation.

In some embodiments, the distal curved portion 718 can move between (i) a relaxed position in which the distal curved portion 718 has the curved shape illustrated in FIG. 7A and (ii) a constrained position in which the distal curved portion 718 is more closely aligned with the longitudinal axis Z (e.g., with the bend angle A reduced). FIG. 7B, for example, is a side view of the of the catheter assembly 610 with a dilator 708 inserted through the valve 602 and through the lumen of the elongate member 614 in accordance with embodiments of the present technology. In the illustrated embodiment, the dilator 708 includes a proximal coupling portion 709 secured to and/or mated to a corresponding portion of the valve 602. The dilator 708 extends entirely through the lumen of the elongate member 614 and out of the distal opening 619. When inserted through the lumen of the elongate member 614, the dilator 708 can constrain the distal curved portion 718 as shown in FIG. 7B to reduce the bend angle A (FIG. 7A) and more closely align the distal curved portion 718 with the longitudinal axis Z (FIG. 7A). Alternatively, a guidewire (not shown) can constrain the distal curved portion 718 to reduce the bend angle A (FIG. 7A) and more closely align the distal curved portion 718 with the longitudinal axis Z (FIG. 7A). In some embodiments, the dilator 708 and/or the valve 702 can be of the type disclosed in U.S. patent application Ser. No. 18/156,944, filed Jan. 19, 2023, and titled “CLOT TREATMENT SYSTEMS WITH DILATOR LOCKING MECHANISMS, AND ASSOCIATED DEVICES AND METHODS,” which is incorporated herein by reference in its entirety.

Referring to FIGS. 7A and 7B, the distal curved portion 718 can be shaped using a heat setting process or other suitable process to have the curved shape illustrated in FIG. 7A or another curved shape (e.g., a full pigtail shape, a Tiger curve shape, a Jacky curve shape, an Amplatz left shape, an LCB shape, an RCB shape, a Judkins left shape, a Judkins right shape, a Multipurpose A2 shape, an IM shape, a 3D LIMA shape, a IM VB -1 shape, and so on.). For example, as is known in the art of heat setting shape memory structures, a fixture, mandrel, or mold may be used to hold the distal curved portion 718 in its desired shape, and then the distal curved portion 718 can be subjected to an appropriate heat treatment such that a shape memory material (e.g., metal, nitinol, steel) structure used to form the elongate member 614 (e.g., a braid, a coil, etc.) assume or are otherwise shape-set to the outer contour of the mandrel or mold. The heat setting process may be performed in an oven or fluidized bed, as is well-known. Therefore, the heat setting process can impart a desired shape, geometry, bend, and/or curve in one or more super-elastic and/or shape memory material or materials used to form the elongate member 614. Accordingly, the distal curved portion 718 may be radially constrained without plastic deformation as shown in FIG. 7B and will self-expand on release of the radial constraint to the position illustrated in FIG. 7A. In some embodiments, the distal curved portion 718 and/or the elongate member 614 can include some features that are at least generally similar in structure and function, or identical in structure and function, to those of the catheters disclosed in U.S. patent application Ser. No. 17/529,018, filed Nov. 17, 2021, and titled “CATHETERS HAVING SHAPED DISTAL PORTIONS, AND ASSOCIATED SYSTEMS AND METHODS,” which is incorporated herein by reference in its entirety.

In some embodiments, the elongate member 614 can have one or more apertures formed at and/or proximal to the distal curved portion 718. FIGS. 8A and 8B, for example, are enlarged side views of a distal portion of the elongate member 614 of the catheter assembly 610 in accordance with embodiments of the present technology. The elongate member 614 has a first size (e.g., 24 French) in FIG. 8A, and a second size (e.g., 16 French) smaller than the first size in FIG. 8B. Referring to FIGS. 8A and 8B, the elongate member 614 includes/defines one or more (e.g., a plurality of) apertures 890 (e.g., holes, side holes) fluidly coupled to the lumen of the elongate member 614. In the illustrated embodiment, the apertures 890 can be circumferentially aligned relative to the longitudinal axis Z (FIG. 6) and spaced apart from one another relative to the longitudinal axis Z proximal of the distal curved portion 718. In other embodiments, some or all of the apertures 890 can be circumferentially distributed, spaced in different manners, formed in/at the distal curved portion 718 and/or other portions of the elongate member 614, etc. Referring to FIGS. 6, 8A, and 8B, when the lumen of the elongate member 614 is aspirated via the aspiration source assembly 620, the aspiration pressure can be applied through the apertures 890 and/or through the distal opening 619. Likewise, when the lumen of the elongate member 614 is irrigated via the irrigation assembly, the irrigation fluid can be injected through the apertures 890 and/or through the distal opening 619. In some aspects of the present technology, the apertures 890 can reduce the likelihood of clogging of the elongate member 614 and reduce the aspiration force from each of the apertures 890 and/or the distal opening 619.

Referring to FIGS. 8A and 8B, in some embodiments the catheter assembly 610 can include a cover or other feature (not shown) that can be manipulated intraprocedurally to cover one or more of the apertures 890. For example, another elongate member, sheath, etc., can be advanced through the lumen of the elongate member 614 and/or over an outer surface of the elongate member 614 to cover the apertures 890. Covering the apertures 890 can increase a resultant aspiration force at the distal opening 619. The cover may include one or more apertures that align with the apertures 890 of the elongate member 614 to selectively control which of apertures 890 through which the resultant aspiration force should be applied. Likewise, some or all of the cover apertures (not shown) can be circumferentially distributed and/or spaced in different manners on the cover. Additionally, another elongate shaft, member, sheath, etc., can be advanced distally through the lumen of the elongate member 614 and/or over an outer surface of the elongate member 614 to more closely align the distal curved portion 718 with the longitudinal axis Z (FIG. 7A). The cover, or elongate shaft, may be operably coupled to a handle (not shown) with an actuator coupled to the elongate shaft. The actuator can be manipulated to move the elongate shaft relative to longitudinal axis Z.

Several aspects of the present technology are set forth in the following examples:

1. A system for aspirating and irrigating a body cavity, comprising:
a catheter assembly comprising—
   an outer elongate member defining an outer lumen; and
   an inner elongate member extending at least partially through the outer elongate member and defining an inner lumen having a distal opening, wherein a distal portion of the outer elongate member is fluidly sealed to the inner elongate member, and wherein the outer elongate member includes an aperture positioned proximal of the distal portion;
an aspiration source fluidly coupled to the inner lumen and configured to aspirate the inner lumen; and
an irrigation source fluidly coupled to the outer lumen and configured to flow an irrigation fluid through the outer lumen and out of the aperture.

2. The system of example 1 wherein the inner elongate member is coaxial with the outer elongate member.

3. The system of example 1 or example 2 wherein the aspiration source is a first syringe, and wherein the irrigation source is a second syringe.

4. The system of any one of examples 1-3 wherein the aperture is one of a plurality of apertures positioned circumferentially about the outer elongate member.

5. The system of any one of examples 1-4, further comprising an aspiration flow control assembly fluidly coupled between the aspiration source and the inner lumen, wherein the aspiration flow control is further fluidly coupled to a waste reservoir, and wherein the aspiration flow control assembly is configured to—
when the aspiration source is actuated in a first manner, permit fluid flow from the inner lumen to the aspiration source while blocking fluid flow from the waste reservoir to the aspiration source, and
when the aspiration source is actuated in a second manner different than the first manner, permit fluid flow from the aspiration source to the waste reservoir while blocking fluid flow from the aspiration source to the inner lumen.

6. The system of example 5 wherein the aspiration source is a syringe having a plunger, wherein the first manner is withdrawal of the plunger, and wherein the second manner is depression of the plunger.

7. The system of any one of examples 1-6, further comprising an irrigation flow control assembly fluidly coupled between the irrigation source and the outer lumen, wherein the irrigation flow control assembly is further fluidly coupled to an irrigation reservoir configured to hold an irrigation fluid, and wherein the irrigation flow control assembly is configured to—
when the irrigation source is actuated in a first manner, permit flow of the irrigation fluid from the irrigation reservoir to the irrigation source while blocking fluid flow from the outer lumen to the irrigation source, and
when the irrigation source is actuated in a second manner different than the first manner, permit the irrigation fluid to flow from the irrigation source into the outer lumen while blocking flow of the irrigation fluid from the irrigation source into the irrigation reservoir.

8. The system of example 7 wherein the irrigation source is a syringe having a plunger, wherein the first manner is withdrawal of the plunger, and wherein the second manner is depression of the plunger.

9. The system of any one of examples 1-8, further comprising:
an aspiration flow control assembly fluidly coupled between the aspiration source and the inner lumen, wherein the aspiration flow control assembly is further fluidly coupled to a waste reservoir, and wherein the aspiration flow control assembly is configured to—
   when the aspiration source is actuated in a first manner, permit fluid flow from the inner lumen to the aspiration source while blocking fluid flow from the waste reservoir to the aspiration source, and
   when the aspiration source is actuated in a second manner different than the first manner, permit fluid flow from the aspiration source to the waste reservoir while blocking fluid flow from the aspiration source to the inner lumen; and
an irrigation flow control assembly fluidly coupled between the irrigation source and the outer lumen, wherein the irrigation flow control assembly is further fluidly coupled to an irrigation reservoir configured to hold an irrigation fluid, and wherein the irrigation flow control assembly is configured to—
   when the irrigation source is actuated in a third manner, permit flow of the irrigation fluid from the irrigation reservoir to the irrigation source while blocking fluid flow from the outer lumen to the irrigation source, and
   when the irrigation source is actuated in a fourth manner different than the third manner, permit the irrigation fluid to flow from the irrigation source into the outer lumen while blocking flow of the irrigation fluid from the irrigation source into the irrigation reservoir.

10. The system of example 9 wherein the aspiration source is a first syringe having a first plunger, wherein the first manner is withdrawal of the first plunger, wherein the second manner is depression of the second plunger, wherein the irrigation source is a second syringe having a second plunger, wherein the third manner is withdrawal of the second plunger, and wherein the fourth manner is depression of the second plunger.

11. The system of example 10 wherein the first plunger and the second plunger are mechanically coupled and configured to move together during withdrawal and depression.

12. The system of any one of examples 1-11, further comprising:
an aspiration flow control assembly fluidly coupled between the aspiration source and the inner lumen, wherein the aspiration flow control assembly comprises:
   a first connector configured to be fluidly coupled to the inner lumen;
   a second connector configured to be fluidly coupled to a waste reservoir;
   a first one-way valve positioned between the first connector and the aspiration source, wherein the first one-way valve is positioned to (a) permit fluid flow through the first connector from the inner lumen to the aspiration source and (b) inhibit fluid flow through the first connector from the aspiration source to the inner lumen; and
   a second one-way valve positioned between the second connector and the aspiration source, wherein the second one-way valve is positioned to (a) permit fluid flow through the second connector from the aspiration source to the waste reservoir and (b) inhibit fluid flow through the second connector from the waste reservoir to aspiration source.

13. The system of example 12 wherein—
the aspiration source comprises a syringe having a plunger;
withdrawal of the plunger is configured to aspirate material from the cavity through the inner lumen;
   during withdrawal of the plunger, the first one-way valve is positioned to permit flow of the material through the first connector into the syringe and the second one-way valve is positioned to inhibit flow from the waste reservoir to the syringe;
depression of the plunger is configured to flow the aspirated material from the syringe to the waste reservoir; and
   during depression of the plunger, the first-one way valve is positioned to inhibit flow of the aspirated material through the first connector into the inner lumen and the second one-way valve is positioned to permit flow of the aspirated material from the syringe through the second connector into the waste reservoir.

14. The system of any one of examples 1-13, further comprising:
an irrigation flow control assembly fluidly coupled between the irrigation source and the outer lumen, wherein the aspiration flow control assembly comprises:
   a first connector configured to be fluidly coupled to the outer lumen;
   a second connector configured to be fluidly coupled to an irrigation reservoir;
   a first one-way valve positioned between the first connector and the irrigation source, wherein the first one-way valve is positioned to (a) permit fluid flow through the first connector from the irrigation source to the outer lumen and (b) inhibit fluid flow through the first connector from the inner lumen to the irrigation source; and
   a second one-way valve positioned between the second connector and the irrigation source, wherein the second one-way valve is positioned to (a) permit fluid flow through the second connector from the irrigation reservoir to the irrigation source and (b) inhibit fluid flow through the second connector from the irrigation source to the irrigation reservoir.

15. The system of example 14 wherein—
the irrigation source comprises a syringe having a plunger;
withdrawal of the plunger is configured to at least partially fill the syringe with an irrigation fluid from the irrigation reservoir;
   during withdrawal of the plunger, the first one-way valve is positioned to inhibit flow through the first connector into the syringe and the second one-way valve is positioned to permit flow of the irrigation fluid from the irrigation reservoir to the syringe;
depression of the plunger is configured to flow the irrigation fluid from the syringe to the outer lumen; and
   during depression of the plunger, the first-one way valve is positioned to permit flow of the irrigation fluid through the first connector into the outer lumen and the second one-way valve is positioned to inhibit flow of the irrigation fluid from the syringe through the second connector into the irrigation reservoir.

16. The system of any one of examples 1-15, further comprising:
an aspiration flow control assembly fluidly coupled between the aspiration source and the inner lumen, wherein the aspiration flow control assembly comprises:
   a first connector configured to be fluidly coupled to the inner lumen;
   a second connector configured to be fluidly coupled to a waste reservoir;
   a first one-way valve positioned between the first connector and the aspiration source, wherein the first one-way valve is positioned to (a) permit fluid flow through the first connector from the inner lumen to the aspiration source and (b) inhibit fluid flow through the first connector from the aspiration source to the inner lumen; and
   a second one-way valve positioned between the second connector and the aspiration source, wherein the second one-way valve is positioned to (a) permit fluid flow through the second connector from the aspiration source to the waste reservoir and (b) inhibit fluid flow through the second connector from the waste reservoir to aspiration source; and
an irrigation flow control assembly fluidly coupled between the irrigation source and the outer lumen, wherein the aspiration flow control assembly comprises:
   a third connector configured to be fluidly coupled to the outer lumen;
   a fourth connector configured to be fluidly coupled to an irrigation reservoir;
   a third one-way valve positioned between the third connector and the irrigation source, wherein the third one-way valve is positioned to (a) permit fluid flow through the third connector from the irrigation source to the outer lumen and (b) inhibit fluid flow through the third connector from the inner lumen to the irrigation source; and
   a fourth one-way valve positioned between the fourth connector and the irrigation source, wherein the fourth one-way valve is positioned to (a) permit fluid flow through the fourth connector from the irrigation reservoir to the irrigation source and (b) inhibit fluid flow through the fourth connector from the irrigation source to the irrigation reservoir.

17. The system of example 16 wherein—
the aspiration source is a first syringe having a first plunger;
   withdrawal of the first plunger is configured to aspirate material from the cavity through the inner lumen;
      during withdrawal of the first plunger, the first one-way valve is positioned to permit flow of the material through the first connector into the first syringe and the second one-way valve is positioned to inhibit flow from the waste reservoir to the first syringe;
   depression of the first plunger is configured to flow the aspirated material from the first syringe to the waste reservoir; and
      during depression of the first plunger, the first-one way valve is positioned to inhibit flow of the aspirated material through the first connector into the inner lumen and the second one-way valve is positioned to permit flow of the aspirated material from the first syringe through the second connector into the waste reservoir;
the irrigation source is a second syringe having a second plunger;
   withdrawal of the second plunger is configured to at least partially fill the second syringe with an irrigation fluid from the irrigation reservoir;
      during withdrawal of the second plunger, the third one-way valve is positioned to inhibit flow through the third connector into the second syringe and the fourth one-way valve is positioned to permit flow of the irrigation fluid from the irrigation reservoir to the second syringe;
   depression of the second plunger is configured to flow the irrigation fluid from the second syringe to the outer lumen; and
      during depression of the second plunger, the third-one way valve is positioned to permit flow of the irrigation fluid through the third connector into the outer lumen and the fourth one-way valve is positioned to inhibit flow of the irrigation fluid from the second syringe through the fourth connector into the irrigation reservoir.

18. The system of example 17 wherein the first plunger and the second plunger are mechanically coupled and configured to move together.

19. The system of any one of examples 1-18 wherein the inner elongate member is a reinforced catheter, and wherein the outer elongate member is a tube formed from a plastic material.

20. A method of treating material within a body cavity of a patient, the method comprising:
percutaneously inserting a catheter assembly into the patient such that the distal portion of the catheter assembly is within the cavity;
aspirating material from the cavity through an inner lumen of the catheter assembly; and
flowing an irrigation fluid through an outer lumen of the catheter assembly, out of an outer aperture in the catheter assembly, and into the cavity to irrigate the cavity, wherein the outer lumen is coaxial with the inner lumen.

21. The method of example 20, further comprising:
inserting a mechanical disruptor element through the inner lumen;
expanding the mechanical disruptor element within the cavity; and
engaging the mechanical disruptor element with the material within the cavity to mechanically disrupt the material.

22. The method of example 20 or example 21 wherein aspirating the material from the cavity comprises activating a syringe fluidly coupled to the inner lumen.

23. The method of any one of examples 20-22 wherein flowing the irrigation fluid through the outer lumen comprises activating a syringe fluidly coupled to the outer lumen.

24. The method of any one of examples 20-23 wherein aspirating the material from the cavity comprises withdrawing a plunger of an aspiration syringe fluidly coupled to the inner lumen, and wherein flowing the irrigation fluid through the outer lumen comprises depressing a plunger of an irrigation syringe fluidly coupled to the outer lumen.

25. The method of example 24 wherein the method further comprises:
withdrawing the plunger of the irrigation syringe to draw the irrigation fluid into the irrigation syringe; and
depressing the plunger of the aspiration syringe to expel the aspirated material from the aspiration syringe.

26. The method of example 25 wherein the method further comprises:
simultaneously withdrawing the plunger of the aspiration syringe to aspirate the material from the cavity and withdrawing the plunger of the irrigation syringe to draw the irrigation fluid into the irrigation syringe; and
simultaneously depressing the plunger of the aspiration syringe to expel the aspirated material from the aspiration syringe and depressing the plunger of the irrigation syringe to flow the irrigation fluid into the cavity to irrigate the cavity.

27. The method of example 26 wherein the plunger of the aspiration syringe and the plunger of the irrigation syringe are mechanically linked to move together.

28. A system for aspirating and irrigating a body cavity, comprising:
a catheter assembly comprising—
   an outer elongate member defining an outer lumen; and
   an inner elongate member extending at least partially through the outer elongate member and defining an inner lumen having a distal opening, wherein a distal portion of the outer elongate member is fluidly sealed to the inner elongate member, and wherein the outer elongate member includes an aperture positioned proximal of the distal portion;
an aspiration syringe fluidly coupled to the inner lumen and configured to aspirate the inner lumen; and
an irrigation syringe fluidly coupled to the outer lumen and configured to flow an irrigation fluid through the outer lumen and out of the aperture.

29. The system of example 28 wherein the aspiration syringe and the irrigation syringe are mechanically linked to be actuated in synchronization.

30. A method of using the system of any one of examples 1-19, 28, or 29 to treat material within a body cavity of a patient.

31. An aspiration flow control assembly for use within an aspiration and irrigation system according to any one of examples 5, 6, 9-13, or 16-18.

32. An irrigation flow control assembly for use within an aspiration and irrigation system according to any one of examples 7-11 or 14-18.

33. A combined aspiration flow control assembly and irrigation flow control assembly for use within an aspiration and irrigation system according to any one of examples 9-11 or 16-18.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for aspirating and irrigating a body cavity, comprising:
a catheter assembly comprising—
an outer elongate member defining an outer lumen; and
an inner elongate member extending at least partially through the outer elongate member and defining an inner lumen having a distal opening, wherein a distal portion of the outer elongate member is fluidly sealed to the inner elongate member, and wherein the outer elongate member includes an aperture positioned proximal of the distal portion;
an aspiration source fluidly coupled to the inner lumen and configured to aspirate the inner lumen; and
an irrigation source fluidly coupled to the outer lumen and configured to flow an irrigation fluid through the outer lumen and out of the aperture.

2. The system of claim 1 wherein the inner elongate member is coaxial with the outer elongate member.

3. The system of claim 1 wherein the aspiration source is a first syringe, and wherein the irrigation source is a second syringe.

4. The system of claim 1 wherein the aperture is one of a plurality of apertures positioned circumferentially about the outer elongate member.

5. The system of any one of claim 1, further comprising an aspiration flow control assembly fluidly coupled between the aspiration source and the inner lumen, wherein the aspiration flow control is further fluidly coupled to a waste reservoir, and wherein the aspiration flow control assembly is configured to—
when the aspiration source is actuated in a first manner, permit fluid flow from the inner lumen to the aspiration source while blocking fluid flow from the waste reservoir to the aspiration source, and
when the aspiration source is actuated in a second manner different than the first manner, permit fluid flow from the aspiration source to the waste reservoir while blocking fluid flow from the aspiration source to the inner lumen.

6. The system of claim 5 wherein the aspiration source is a syringe having a plunger, wherein the first manner is withdrawal of the plunger, and wherein the second manner is depression of the plunger.

7. The system of claim 1, further comprising an irrigation flow control assembly fluidly coupled between the irrigation source and the outer lumen, wherein the irrigation flow control assembly is further fluidly coupled to an irrigation reservoir configured to hold an irrigation fluid, and wherein the irrigation flow control assembly is configured to—
when the irrigation source is actuated in a first manner, permit flow of the irrigation fluid from the irrigation reservoir to the irrigation source while blocking fluid flow from the outer lumen to the irrigation source, and
when the irrigation source is actuated in a second manner different than the first manner, permit the irrigation fluid to flow from the irrigation source into the outer lumen while blocking flow of the irrigation fluid from the irrigation source into the irrigation reservoir.

8. The system of claim 7 wherein the irrigation source is a syringe having a plunger, wherein the first manner is withdrawal of the plunger, and wherein the second manner is depression of the plunger.

9. The system of claim 1, further comprising:
an aspiration flow control assembly fluidly coupled between the aspiration source and the inner lumen, wherein the aspiration flow control assembly is further fluidly coupled to a waste reservoir, and wherein the aspiration flow control assembly is configured to—
when the aspiration source is actuated in a first manner, permit fluid flow from the inner lumen to the aspiration source while blocking fluid flow from the waste reservoir to the aspiration source, and
when the aspiration source is actuated in a second manner different than the first manner, permit fluid flow from the aspiration source to the waste reservoir while blocking fluid flow from the aspiration source to the inner lumen; and
an irrigation flow control assembly fluidly coupled between the irrigation source and the outer lumen, wherein the irrigation flow control assembly is further fluidly coupled to an irrigation reservoir configured to hold an irrigation fluid, and wherein the irrigation flow control assembly is configured to—
when the irrigation source is actuated in a third manner, permit flow of the irrigation fluid from the irrigation reservoir to the irrigation source while blocking fluid flow from the outer lumen to the irrigation source, and
when the irrigation source is actuated in a fourth manner different than the third manner, permit the irrigation fluid to flow from the irrigation source into the outer lumen while blocking flow of the irrigation fluid from the irrigation source into the irrigation reservoir.

10. The system of claim 9 wherein the aspiration source is a first syringe having a first plunger, wherein the first manner is withdrawal of the first plunger, wherein the second manner is depression of the second plunger, wherein the irrigation source is a second syringe having a second plunger, wherein the third manner is withdrawal of the second plunger, and wherein the fourth manner is depression of the second plunger.

11. The system of claim 10 wherein the first plunger and the second plunger are mechanically coupled and configured to move together during withdrawal and depression.

12. The system of claim 1, further comprising:
an aspiration flow control assembly fluidly coupled between the aspiration source and the inner lumen, wherein the aspiration flow control assembly comprises:
a first connector configured to be fluidly coupled to the inner lumen;
a second connector configured to be fluidly coupled to a waste reservoir;
a first one-way valve positioned between the first connector and the aspiration source, wherein the first one-way valve is positioned to (a) permit fluid flow through the first connector from the inner lumen to the aspiration source and (b) inhibit fluid flow through the first connector from the aspiration source to the inner lumen; and
a second one-way valve positioned between the second connector and the aspiration source, wherein the second one-way valve is positioned to (a) permit fluid flow through the second connector from the aspiration source to the waste reservoir and (b) inhibit fluid flow through the second connector from the waste reservoir to aspiration source.

13. The system of claim 12 wherein—
the aspiration source comprises a syringe having a plunger;
withdrawal of the plunger is configured to aspirate material from the cavity through the inner lumen;
during withdrawal of the plunger, the first one-way valve is positioned to permit flow of the material through the first connector into the syringe and the second one-way valve is positioned to inhibit flow from the waste reservoir to the syringe;
depression of the plunger is configured to flow the aspirated material from the syringe to the waste reservoir; and
during depression of the plunger, the first-one way valve is positioned to inhibit flow of the aspirated material through the first connector into the inner lumen and the second one-way valve is positioned to permit flow of the aspirated material from the syringe through the second connector into the waste reservoir.

14. The system of claim 1, further comprising:
an irrigation flow control assembly fluidly coupled between the irrigation source and the outer lumen, wherein the aspiration flow control assembly comprises:
a first connector configured to be fluidly coupled to the outer lumen;
a second connector configured to be fluidly coupled to an irrigation reservoir;
a first one-way valve positioned between the first connector and the irrigation source, wherein the first one-way valve is positioned to (a) permit fluid flow through the first connector from the irrigation source to the outer lumen and (b) inhibit fluid flow through the first connector from the inner lumen to the irrigation source; and
a second one-way valve positioned between the second connector and the irrigation source, wherein the second one-way valve is positioned to (a) permit fluid flow through the second connector from the irrigation reservoir to the irrigation source and (b) inhibit fluid flow through the second connector from the irrigation source to the irrigation reservoir.

15. The system of claim 14 wherein—
the irrigation source comprises a syringe having a plunger;
withdrawal of the plunger is configured to at least partially fill the syringe with an irrigation fluid from the irrigation reservoir;
during withdrawal of the plunger, the first one-way valve is positioned to inhibit flow through the first connector into the syringe and the second one-way valve is positioned to permit flow of the irrigation fluid from the irrigation reservoir to the syringe;
depression of the plunger is configured to flow the irrigation fluid from the syringe to the outer lumen; and
during depression of the plunger, the first-one way valve is positioned to permit flow of the irrigation fluid through the first connector into the outer lumen and the second one-way valve is positioned to inhibit flow of the irrigation fluid from the syringe through the second connector into the irrigation reservoir.

16. The system of claim 1, further comprising:
an aspiration flow control assembly fluidly coupled between the aspiration source and the inner lumen, wherein the aspiration flow control assembly comprises:
a first connector configured to be fluidly coupled to the inner lumen;
a second connector configured to be fluidly coupled to a waste reservoir;
a first one-way valve positioned between the first connector and the aspiration source, wherein the first one-way valve is positioned to (a) permit fluid flow through the first connector from the inner lumen to the aspiration source and (b) inhibit fluid flow through the first connector from the aspiration source to the inner lumen; and
a second one-way valve positioned between the second connector and the aspiration source, wherein the second one-way valve is positioned to (a) permit fluid flow through the second connector from the aspiration source to the waste reservoir and (b) inhibit fluid flow through the second connector from the waste reservoir to aspiration source; and
an irrigation flow control assembly fluidly coupled between the irrigation source and the outer lumen, wherein the aspiration flow control assembly comprises:
a third connector configured to be fluidly coupled to the outer lumen;
a fourth connector configured to be fluidly coupled to an irrigation reservoir;
a third one-way valve positioned between the third connector and the irrigation source, wherein the third one-way valve is positioned to (a) permit fluid flow through the third connector from the irrigation source to the outer lumen and (b) inhibit fluid flow through the third connector from the inner lumen to the irrigation source; and
a fourth one-way valve positioned between the fourth connector and the irrigation source, wherein the fourth one-way valve is positioned to (a) permit fluid flow through the fourth connector from the irrigation reservoir to the irrigation source and (b) inhibit fluid flow through the fourth connector from the irrigation source to the irrigation reservoir.

17. The system of claim 16 wherein—
the aspiration source is a first syringe having a first plunger;
withdrawal of the first plunger is configured to aspirate material from the cavity through the inner lumen;
during withdrawal of the first plunger, the first one-way valve is positioned to permit flow of the material through the first connector into the first syringe and the second one-way valve is positioned to inhibit flow from the waste reservoir to the first syringe;
depression of the first plunger is configured to flow the aspirated material from the first syringe to the waste reservoir; and
during depression of the first plunger, the first-one way valve is positioned to inhibit flow of the aspirated material through the first connector into the inner lumen and the second one-way valve is positioned to permit flow of the aspirated material from the first syringe through the second connector into the waste reservoir;
the irrigation source is a second syringe having a second plunger;
withdrawal of the second plunger is configured to at least partially fill the second syringe with an irrigation fluid from the irrigation reservoir;
during withdrawal of the second plunger, the third one-way valve is positioned to inhibit flow through the third connector into the second syringe and the fourth one-way valve is positioned to permit flow of the irrigation fluid from the irrigation reservoir to the second syringe;
depression of the second plunger is configured to flow the irrigation fluid from the second syringe to the outer lumen; and
during depression of the second plunger, the third-one way valve is positioned to permit flow of the irrigation fluid through the third connector into the outer lumen and the fourth one-way valve is positioned to inhibit flow of the irrigation fluid from the second syringe through the fourth connector into the irrigation reservoir.

18. The system of claim 17 wherein the first plunger and the second plunger are mechanically coupled and configured to move together.

19. The system of claim 1 wherein the inner elongate member is a reinforced catheter, and wherein the outer elongate member is a tube formed from a plastic material.

20. A method of treating material within a body cavity of a patient, the method comprising:
percutaneously inserting a catheter assembly into the patient such that the distal portion of the catheter assembly is within the cavity;
aspirating material from the cavity through an inner lumen of the catheter assembly; and
flowing an irrigation fluid through an outer lumen of the catheter assembly, out of an outer aperture in the catheter assembly, and into the cavity to irrigate the cavity, wherein the outer lumen is coaxial with the inner lumen.

21. The method of claim 20, further comprising:
inserting a mechanical disruptor element through the inner lumen;
expanding the mechanical disruptor element within the cavity; and
engaging the mechanical disruptor element with the material within the cavity to mechanically disrupt the material.

22. The method of claim 20 wherein aspirating the material from the cavity comprises activating a syringe fluidly coupled to the inner lumen.

23. The method of claim 20 wherein flowing the irrigation fluid through the outer lumen comprises activating a syringe fluidly coupled to the outer lumen.

24. The method of claim 20 wherein aspirating the material from the cavity comprises withdrawing a plunger of an aspiration syringe fluidly coupled to the inner lumen, and wherein flowing the irrigation fluid through the outer lumen comprises depressing a plunger of an irrigation syringe fluidly coupled to the outer lumen.

25. The method of claim 24 wherein the method further comprises:
withdrawing the plunger of the irrigation syringe to draw the irrigation fluid into the irrigation syringe; and
depressing the plunger of the aspiration syringe to expel the aspirated material from the aspiration syringe.

26. The method of claim 25 wherein the method further comprises:
simultaneously withdrawing the plunger of the aspiration syringe to aspirate the material from the cavity and withdrawing the plunger of the irrigation syringe to draw the irrigation fluid into the irrigation syringe; and
simultaneously depressing the plunger of the aspiration syringe to expel the aspirated material from the aspiration syringe and depressing the plunger of the irrigation syringe to flow the irrigation fluid into the cavity to irrigate the cavity.

27. The method of claim 26 wherein the plunger of the aspiration syringe and the plunger of the irrigation syringe are mechanically linked to move together.

28. A system for aspirating and irrigating a body cavity, comprising:
a catheter assembly comprising—
an outer elongate member defining an outer lumen; and
an inner elongate member extending at least partially through the outer elongate member and defining an inner lumen having a distal opening, wherein a distal portion of the outer elongate member is fluidly sealed to the inner elongate member, and wherein the outer elongate member includes an aperture positioned proximal of the distal portion;
an aspiration syringe fluidly coupled to the inner lumen and configured to aspirate the inner lumen; and
an irrigation syringe fluidly coupled to the outer lumen and configured to flow an irrigation fluid through the outer lumen and out of the aperture.

29. The system of claim 28 wherein the aspiration syringe and the irrigation syringe are mechanically linked to be actuated in synchronization.
